(19) United States
(12) Patent Application Publication
(10) Pub. No.: US 2019/0022165 A1
(43) Pub. Date: Jan. 24, 2019

(54) METHODS AND COMPOSITIONS FOR THE TREATMENT AND PREVENTION OF PULMONARY ARTERIAL HYPERTENSION

(71) Applicant: STEALTH BIOTHERAPEUTICS CORP, MONACO (MC)

(72) Inventor: D. Travis WILSON, Newton, MA (US)

(21) Appl. No.: 16/081,364

(22) PCT Filed: Mar. 2, 2017
(86) PCT No.: PCT/US2017/020400

§ 371 Date: Aug. 30, 2018

Related U.S. Application Data
(60) Provisional application No. 62/302,393, filed on Mar. 2, 2016.

(51) Int. Cl. A61K 38/07; A61K 9/00; A61P 9/12; A61B 5/021

(52) U.S. Cl. CPC A61K 38/07; A61K 9/0073; A61K 9/0053; A61K 9/0019; A61K 45/06; A61P 9/12; A61B 5/021; A61K 2300/00; A61K 9/0014

(57) ABSTRACT

The disclosure provides methods of preventing or treating pulmonary arterial hypertension (PAH) in a mammalian subject, reducing risk factors associated with PAH, and/or reducing the likelihood or severity of PAH. The methods comprise administering to the subject an effective amount of an aromatic-cationic peptide.

### CROSS REFERENCE TO RELATED APPLICATION

This application claims priority to U.S. Provisional Application 62/302,393, filed Mar. 2, 2016, the entire content of which is incorporated herein by reference in its entirety.

### TECHNICAL FIELD

The present technology relates generally to compositions and methods for preventing, ameliorating, or treating pulmonary arterial hypertension (PAH) and/or reducing the severity of one or more risk factors, signs, or symptoms associated with PAH. Additionally, the present technology relates to administering an effective amount of an aromatic-cationic peptide, such as 2′6′-Dmt-D-Arg-Phe-Lys-NH₂, Phe-D-Arg-Phe-Lys-NH₂, or D-Arg-2′6′-Dmt-Lys-Phe-NH₂, to a subject suffering from or at risk for PAH.

### BACKGROUND

The following description is provided to assist the understanding of the reader. None of the information provided or references cited is admitted to be prior art to the compositions and methods disclosed herein.

Pulmonary hypertension (PH) is a lung disorder in which mean pulmonary arterial pressure rises above normal levels (25 mm Hg at rest and 30 mm Hg during exercise). PH is classified into arterial, venous, hypoxic, thromboembolitic, and miscellaneous varieties. Of these varieties of PH, pulmonary arterial hypertension (PAH) is typically associated with the worst prognosis. PAH is subclassified as idiopathic PAH (IPAH), familial PAH (FPAH), and associated PAH (APAH) varieties.

Pulmonary arterial hypertension (PAH) is a chronic and progressive disease of the lung vascular system in which endothelial dysfunction and vascular remodeling of endothelial and smooth muscle cells lead to the obstruction of pulmonary arteries, resulting in increased pulmonary vascular resistance and pulmonary arterial pressures. This leads to reduced cardiac output, right ventricular failure (cor pulmonale), and ultimately death within two to three years of diagnosis, if untreated.

In the United States, the estimated incidence and prevalence of PAH are 2.3 and 12.4 cases per million adults, respectively. PAH can develop in men and women at any age, but the disorder is nearly twice as common in females as in males. Despite recent advances in elucidating potential molecular pathways implicated in PAH and therapeutic approaches that appear to prolong survival in some PAH patients, the prognosis of PAH remains poor and there is no cure for this disorder.

### SUMMARY

In one aspect, the present disclosure provides methods for treating or preventing pulmonary arterial hypertension (PAH), and/or treating or preventing the signs or symptoms of PAH in a subject in need thereof comprising administering to the subject a therapeutically effective amount of an aromatic-cationic peptide such as 2′6′-Dmt-D-Arg-Phe-Lys-NH₂, Phe-D-Arg-Phe-Lys-NH₂, or D-Arg-2′6′-Dmt-Lys-Phe-NH₂ or a pharmaceutically acceptable salt thereof. In some embodiments of the methods of the present technology, the pharmaceutically acceptable salt comprises acetate, tartrate, or trifluoroacetate salt.

In one aspect, the present technology provides for methods for reducing the risk, signs or symptoms of PAH in a subject in need thereof. In some embodiments, the method includes administering to the subject a therapeutically effective amount of the aromatic-cationic peptide 2′6′-Dmt-D-Arg-Phe-Lys-NH₂, Phe-D-Arg-Phe-Lys-NH₂, or D-Arg-2′6′-Dmt-Lys-Phe-NH₂ or a pharmaceutically acceptable salt thereof, thereby resulting in the prevention or delay of onset of one or more risks, signs or symptoms of PAH. In some embodiments of the methods of the present technology, the pharmaceutically acceptable salt comprises acetate, tartrate, or trifluoroacetate salt.

In some embodiments of the methods of the present technology, the signs or symptoms of PAH include one or more of persistent dyspnea on exertion, chest pain, light-headedness, exertional presyncope/syncope, palpitations, fatigue, weakness, hoarseness in the voice due to compression of the left laryngeal nerve by the dilated pulmonary artery, venous jugular distension, hepato-jugular reflux, hepatomegaly, hepatalgia, lower limb edema, ascites, generalized edema, intimal fibrosis of pulmonary arteries, increased medial thickness of pulmonary arteries, intimal hyperplasia of muscular pulmonary arteries, pulmonary artery thrombotic lesions, pulmonary arteriolar occlusion, pulmonary vascular pruning, plexiform lesions in pulmonary arteries, elevated serum or plasma brain natriuretic peptide (BNP) (≥180 pg/mL), and elevated serum or plasma N-terminal fragment of proBNP (NT-proBNP) (≥1400 pg/mL).

In some embodiments of the methods of the present technology, the subject displays elevated blood levels of brain natriuretic peptide (BNP) and/or N-terminal fragment of proBNP (NT-proBNP) compared to a normal control subject. In certain embodiments of the methods of the present technology, treatment with the aromatic-cationic peptide normalizes BNP and/or NT-proBNP blood levels.

In some embodiments of the methods of the present technology, the subject harbors a mutation in the bone morphogenetic protein receptor 2 (BMPR²) gene.

In some embodiments of the methods of the present technology, the subject is human.

In some embodiments of the methods of the present technology, the aromatic-cationic peptide is administered orally, topically, systemically, intravenously, subcutaneously, transdermally, iontophoretically, intranasally, intraperitoneally, intramuscularly, or by pulmonary inhalation.

In some embodiments of the methods of the present technology, the aromatic-cationic peptide is administered daily for 1 week or more. In some embodiments of the methods of the present technology, the aromatic-cationic peptide is administered daily for 2 weeks or more. In some embodiments of the methods of the present technology, the aromatic-cationic peptide is administered daily for 3 weeks or more. In some embodiments of the methods of the present technology, the aromatic-cationic peptide is administered daily for 4 weeks or more. In some embodiments of the methods of the present technology, the aromatic-cationic peptide is administered daily for 6 weeks or more. In some embodiments of the methods of the present technology, the aromatic-cationic peptide is administered daily for 12 weeks or more.

In some embodiments, in addition to the administration of the aromatic-cationic peptide, the method further comprises separately, sequentially, or simultaneously administering to the subject one or more additional therapeutic agents selected from the group consisting of: endothelin receptor antagonists (ETRAs), guanylate cyclase stimulators, prostacyclin analogues, phosphodiesterase (PDE)-5 inhibitors, dehydroepiandrosterone (DHEA), cyclosporine, tacrolimums, bestatin, imatinib, calcium-channel blockers (CCBs), dichloroacetate (DCA), trimetazidine, ranolazine, 4-phenylbutyrate, tauroursodeoxycholic acid, and salubrinal. In some embodiments of the methods of the present technology, there is a synergistic effect between the aromatic-cationic peptide and the additional therapeutic agent with respect to the prevention or treatment of PAH.

In some embodiments, in addition to the administration of the aromatic-cationic peptide, the method further comprises separately, sequentially, or simultaneously administering to the subject one or more endothelin receptor antagonists (ERTAs) selected from the group consisting of bosentan, ambrisentan, macitentan, and sitaxsentan. In some embodiments of the methods of the present technology, there is a synergistic effect between the aromatic-cationic peptide and the ERTAs with respect to the prevention or treatment of PAH.

In some embodiments, in addition to the administration of the aromatic-cationic peptide, the method further comprises separately, sequentially, or simultaneously administering to the subject one or more prostacyclin analogues selected from the group consisting of epoprostenal, treprostinil, and iloprost. In some embodiments of the methods of the present technology, there is a synergistic effect between the aromatic-cationic peptide and the prostacyclin analogues with respect to the prevention or treatment of PAH.

In some embodiments, in addition to the administration of the aromatic-cationic peptide, the method further comprises separately, sequentially, or simultaneously administering to the subject one or more soluble guanylate cyclase stimulators selected from the group consisting of riociguat and cinaciguat. In some embodiments of the methods of the present technology, there is a synergistic effect between the aromatic-cationic peptide and the soluble guanylate cyclase stimulators with respect to the prevention or treatment of PAH.

In some embodiments, in addition to the administration of the aromatic-cationic peptide, the method further comprises separately, sequentially, or simultaneously administering to the subject one or more phosphodiesterase (PDE)-5 inhibitors selected from the group consisting of sildenafil, tadalafil, and vardenafil. In some embodiments of the methods of the present technology, there is a synergistic effect between the aromatic-cationic peptide and the PDE-5 inhibitors with respect to the prevention or treatment of PAH.

In some embodiments, in addition to the administration of the aromatic-cationic peptide, the method further comprises separately, sequentially or simultaneously administering to the subject one or more calcium-channel blockers (CCBs) selected from the group consisting of nifedipine, diltiazem, and amlodipine. In some embodiments of the methods of the present technology, there is a synergistic effect between the aromatic-cationic peptide and the CCBs with respect to the prevention or treatment of PAH.

In one aspect, the present technology provides for methods for reducing the risk, signs or symptoms of PAH in a mammalian subject. In some embodiments, the method includes administering to the subject a therapeutically effective amount of the aromatic-cationic peptide 2′6′-Dmt-D-Arg-Phe-Lys-NH₂, Phe-D-Arg-Phe-Lys-NH₂, or D-Arg-2′6′-Dmt-Lys-Phe-NH₂ or a pharmaceutically acceptable salt thereof, thereby resulting in the prevention or delay of onset of one or more risks, signs or symptoms of PAH. In some embodiments of the methods of the present technology, the pharmaceutically acceptable salt comprises acetate, tartrate, or trifluoroacetate salt.

In some embodiments of the methods of the present technology, the signs or symptoms of PAH include one or more of persistent dyspnea on exertion, chest pain, light-headedness, exertional presyncope/syncope, palpitations, fatigue, weakness, hoarseness in the voice due to compression of the left laryngeal nerve by the dilated pulmonary artery, venous jugular distension, hepato jugular reflux, hepatomegaly, hepatalgia, lower limb edema, ascites, generalized edema, intimal fibrosis of pulmonary arteries, increased medial thickness of pulmonary arteries, intimal hyperplasia of muscular pulmonary arteries, pulmonary artery thrombotic lesions, pulmonary arteriolar occlusion, pulmonary vascular pruning, plexiform lesions in pulmonary arteries, elevated serum or plasma brain natriuretic peptide (BNP) (≥180 pg/mL), or elevated serum or plasma N-terminal fragment of proBNP (NT-proBNP) (≥1400 pg/mL).

### BRIEF DESCRIPTION OF THE DRAWINGS

FIGS. 1A and 1B are charts showing the right ventricular systolic pressure (RVSP) (mmHg) change from baseline in Cohort 2 and Cohort 3 subjects.

FIG. 2 is a chart showing the right ventricular (RV) fractional area (%) change from baseline (CFB) in Cohort 3 subjects.

### DETAILED DESCRIPTION

It is to be appreciated that certain aspects, modes, embodiments, variations and features of the present technology are described below in various levels of detail in order to provide a substantial understanding of the present technology. The definitions of certain terms as used in this specification are provided below. Unless defined otherwise, all technical and scientific terms used herein generally have the same meaning as commonly understood by one of ordinary skill in the art to which this present technology belongs.

As used in this specification and the appended claims, the singular forms “a”, “an” and “the” include plural referents unless the content clearly dictates otherwise. For example, reference to “a cell” includes a combination of two or more cells, and the like.

As used herein, the “administration” of an agent, drug, or peptide to a subject includes any route of introducing or delivering to a subject a compound to perform its intended function. Administration can be carried out by any suitable route, including orally, intranasally, parenterally (intravenously, intramuscularly, intraperitoneally, or subcutaneously), topically, or by inhalation. Administration includes self-administration and the administration by another.

As used herein, the term “amino acid” includes naturally-occurring amino acids and synthetic amino acids, as well as amino acid analogs and amino acid mimetics that function in a manner similar to the naturally-occurring amino acids. Naturally-occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally-occurring amino acid, i.e., an a-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally-occurring amino acid. Amino acid mimetics refers to chemical compounds that have a structure that is different from the general chemical structure of an amino acid, but that functions in a manner similar to a naturally-occurring amino acid. Amino acids can be referred to herein by either their commonly known three letter symbols or by the one-letter symbols recommended by the IUPAC-IUB Biochemical Nomenclature Commission.

As used herein, the term “effective amount” refers to a quantity sufficient to achieve a desired therapeutic and/or prophylactic effect, e.g., an amount which results in partial or full amelioration of one or more symptoms of pulmonary arterial hypertension (PAH). In the context of therapeutic or prophylactic applications, in some embodiments, the amount of a composition administered to the subject will depend on the type, degree, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, aromatic-cationic peptides, such as 2′6′-Dmt-D-Arg-Phe-Lys-NH₂, Phe-D-Arg-Phe-Lys-NH₂, or D-Arg-2′6′-Dmt-Lys-Phe-NH₂, or a pharmaceutically acceptable salt thereof, such as acetate, tartrate, or trifluoroacetate salt, may be administered to a subject having one or more signs, symptoms, or risk factors of PAH, including, but not limited to, persistent dyspnea on exertion, chest pain, light-headedness, exertional presyncope/syncope, palpitations, fatigue, weakness, hoarseness in the voice due to compression of the left laryngeal nerve by the dilated pulmonary artery, venous jugular distension, hepato jugular reflux, hepatomegaly, hepatalgia, lower limb edema, ascites, generalized edema, intimal fibrosis of pulmonary arteries, increased medial thickness of pulmonary arteries, intimal hyperplasia of muscular pulmonary arteries, pulmonary artery thrombotic lesions, pulmonary arteriolar occlusion, pulmonary vascular pruning, plexiform lesions in pulmonary arteries, elevated serum or plasma brain natriuretic peptide (BNP) (>180 pg/mL), elevated serum or plasma N-terminal fragment of proBNP (NT-proBNP) (>1400 pg/mL), connective tissue disease, portal hypertension, HIV infection, schistosomiasis, pulmonary capillary hemangiomatosis, and exposure to drugs and/or toxins, such as aminorex, fenfluramin derivatives, benfluorex, toxic rapeseed oil, dasatinib, type 1 interferons, L-tryptophan, methamphetamine, and cocaine. For example, a “therapeutically effective amount” of the aromatic-cationic peptides includes levels at which the presence, frequency, or severity of one or more signs, symptoms, or risk factors of PAH are, at a minimum, ameliorated. In some embodiments, a therapeutically effective amount reduces or ameliorates the physiological effects of PAH, and/or the risk factors of PAH, and/or the likelihood of developing PAH. A therapeutically effective amount can be given in one or more administrations.

As used herein, “isolated” or “purified” polypeptide or peptide refers to a polypeptide or peptide that is substantially free of cellular material or other contaminating polypeptides from the cell or tissue source from which the agent is derived, or substantially free from chemical precursors or other chemicals when chemically synthesized. For example, an isolated aromatic-cationic peptide would be free of materials that would interfere with diagnostic or therapeutic uses of the agent. Such interfering materials may include enzymes, hormones and other proteinaceous and nonproteinaceous solutes.

As used herein, the terms “polypeptide,” “peptide,” and “protein” are used interchangeably herein to mean a polymer comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well known in the art.

As used herein, the term “pulmonary arterial hypertension” or “PAH” refers to a disorder characterized by a mean pulmonary arterial pressure (mPAP) greater than or equal to 25 mm Hg at rest or greater than or equal to 30 mm Hg with exercise, with normal pulmonary artery occlusion pressure (i.e., pulmonary-capillary wedge pressure or left ventricular end-diastolic pressure) of less than or equal to 15 mm Hg, in humans. The vascular resistance is at the pulmonary arterioles and capillaries and this defines patients with PAH. If untreated, PAH results in “right heart disease” (also known as “cor pulmonale,” “right ventricular failure,” or “pulmonary heart disease”), which is a term that describes disease of the right-sided cardiac chambers caused by pulmonary arterial hypertension. Excluded from this definition are cases of pulmonary hypertension caused by left ventricular failure of other primary diseases of the left side of the heart and of pulmonary hypertension caused by congenital heart disease.

As used herein, the terms “subject” and “patient” are used interchangeably.

As used herein, the term “simultaneous” therapeutic use refers to the administration of at least two active ingredients by the same route and at the same time or at substantially the same time.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

A “synergistic therapeutic effect” refers to a greater-than-additive therapeutic effect which is produced by a combination of at least two therapeutic agents, and which exceeds that which would otherwise result from the individual administration of the agents. For example, lower doses of one or more therapeutic agents may be used in treating PAH, resulting in increased therapeutic efficacy and decreased side-effects.

“Treating” or “treatment” as used herein covers the treatment of PAH, in a subject, such as a human, and includes: (i) inhibiting PAH, i.e., arresting its development; (ii) relieving PAH, i.e., causing regression of the disorder; (iii) slowing the progression of PAH; and/or (iv) inhibiting, relieving, or slowing progression of one or more symptoms of PAH.

As used herein, “preventing” or “prevention” of a disorder or condition refers to a compound that reduces the occurrence of the disorder or condition in the treated sample relative to an untreated control sample, or delays the onset of one or more symptoms of the disorder or condition relative to the untreated control sample. As used herein, preventing PAH preventing or delaying the initiation of PAH. As used herein, prevention of PAH also includes preventing a recurrence of one or more signs or symptoms of PAH.

It is also to be appreciated that the various modes of treatment or prevention of medical conditions as described herein are intended to mean “substantial,” which includes total but also less than total treatment or prevention, and wherein some biologically or medically relevant result is achieved. The treatment may be a continuous prolonged treatment for a chronic disease or a single, or few time administrations for the treatment of an acute condition.

### Aromatic-Cationic Peptides

The aromatic-cationic peptides of the present technology preferably include a minimum of three amino acids, covalently joined by peptide bonds.

The maximum number of amino acids present in the aromatic-cationic peptides of the present technology is about twenty amino acids covalently joined by peptide bonds. In some embodiments, the total number of amino acids is about twelve. In some embodiments, the total number of amino acids is about nine. In some embodiments, the total number of amino acids is about six. In some embodiments, the total number of amino acids is four.

In some aspects, the present technology provides an aromatic-cationic peptide or a pharmaceutically acceptable salt thereof such as acetate salt, tartrate salt, fumarate salt, hydrochloride salt, or trifluoroacetate salt. In some embodiments, the peptide comprises at least one net positive charge; a minimum of three amino acids; a maximum of about twenty amino acids;

a relationship between the minimum number of net positive charges (pₘ) and the total number of amino acid residues (r) wherein 3pₘ is the largest number that is less than or equal to r+1; and

a relationship between the minimum number of aromatic groups (a) and the total number of net positive charges (pₜ) wherein 2a is the largest number that is less than or equal to pₜ+1, except that when a is 1, pₜ may also be 1.

In some embodiments, the peptide is defined by Formula I:

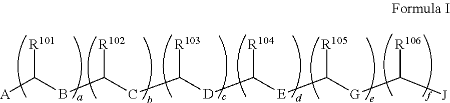

wherein:

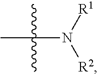

one of A and J is

and the other of A and J is

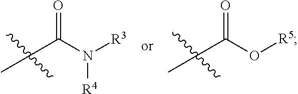

B, C, D, E, and G are each

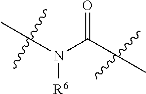

or B, C, D, E, and G are each

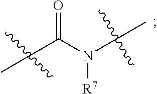

with the proviso that when
   f is 0 and J is not a terminal group, the terminal group is one of G, E, D or C, such that
   one of A and the terminal group is

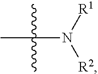

and
the other of A and the terminal group is

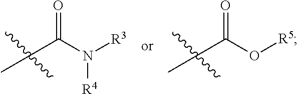

R¹⁰¹ is

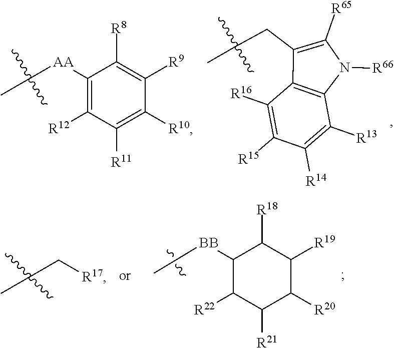

R¹⁰² is

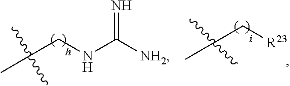

or hydrogen;

R¹⁰³ is

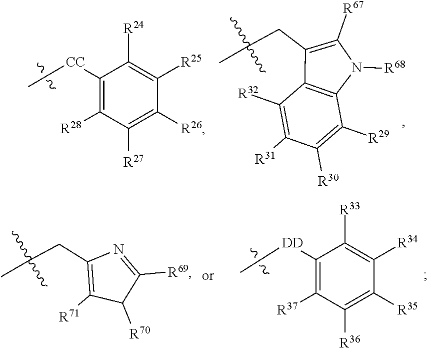

R¹⁰⁴ is

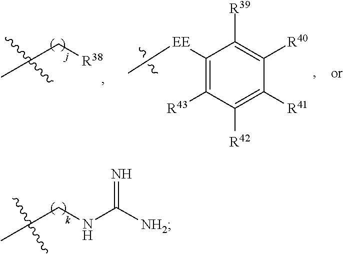

R¹⁰⁵ is

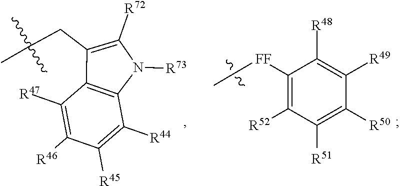

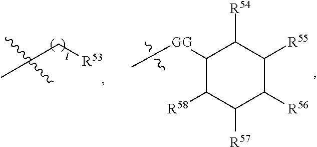

or hydrogen;

R¹⁰⁶ is

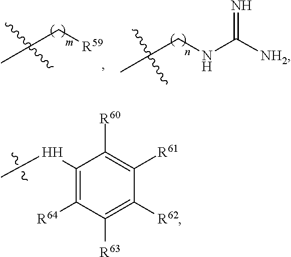

or hydrogen;
provided that when R¹⁰², R¹⁰⁴, and R¹⁰⁶ are identical, then R¹⁰¹, R¹⁰³, and R¹⁰⁵ are not identical;
wherein
   R¹, R², R³, R⁴, and R⁵ are each independently a hydrogen or substituted or unsubstituted C₁-C₆ alkyl, C₂-C₆ alkenyl, C₂-C₆ alkynyl, saturated or unsaturated cycloalkyl, cycloalkylalkyl, aryl, aralkyl, 5- or 6-membered saturated or unsaturated heterocylyl, heteroaryl, or amino protecting group; or R¹ and R² together form a 3, 4, 5, 6, 7, or 8 membered substituted or unsubstituted heterocycyl ring;
   R⁶ and R⁷ at each occurrence are independently a hydrogen or substituted or unsubstituted C₁-C₆ alkyl group;
   R⁸, R⁹, R¹⁰, R¹¹, R¹², R¹³, R¹⁴, R¹⁵, R¹⁶, R¹⁸, R¹⁹, R²⁰, R²¹, R²², R²⁴, R²⁵, R²⁶, R²⁷, R²⁸, R²⁹, R³⁰, R³¹, R³², R³³, R³⁴, R³⁵, R³⁶, R³⁷, R³⁹, R⁴⁰, R⁴¹, R⁴², R⁴³, R⁴⁴, R⁴⁵, R⁴⁶, R⁴⁷, R⁴⁸, R⁴⁹, R⁵⁰, R⁵¹, R⁵², R⁵⁴, R⁵⁵, R^{56 R}⁵⁷, R⁵⁸, R⁶⁰, R⁶¹, R⁶², R⁶³, R⁶⁴, R⁶⁵, R⁶⁷, R⁶⁹, R⁷¹, and R⁷², are each independently a hydrogen, amino, amido, —NO₂, —CN, —OR^{a}, —SR^{a}, —NR^{a}R^{a}, —F, —Cl, —Br, —I, or a substituted or unsubstituted C₁-C₆ alkyl, C₁-C₆ alkoxy, —C(O)-alkyl, —C(O)-aryl, —C(O)-aralkyl, —C(O)₂R^{a}, C₁-C₄ alkylamino, C₁-C₄ dialkylamino, or perhaloalkyl group;
   R⁶⁶, R⁶⁸, R⁷⁰, and R⁷³ are each independently a hydrogen or substituted or unsubstituted C₁-C₆ alkyl group;
   R¹⁷, R²³, R³⁸, R⁵³, and R⁵⁹ are each independently a hydrogen, —OR^{a}, —SR^{a}, —NR^{a}R^{a}, —NR^{a}R^{b}, —CO₂R^{a}, —(CO)NR^{a}R^{a}, —NR^{a}(CO)R^{a}, —NR^{a}C(NH)NH₂, —NR^{a}-dansyl, or a substituted or unsubstituted alkyl, aryl, or aralkyl group;
   AA, BB, CC, DD, EE, FF, GG, and HH are each independently absent, —NH(CO)—, or —CH₂—;
   R^{a} at each occurrence is independently a hydrogen or a substituted or unsubstituted C₁-C₆ alkyl group;
   R^{b} at each occurrence is independently a C₁-C₆ alkylene-NR^{a}-dansyl or C₁-C₆ alkylene-NR^{a}-anthraniloyl group;
   a, b, c, d, e, and fare each independently 0 or 1, with the proviso that a+b+c+d+e+f≥2;
   g, h, k, m, and n are each independently 1, 2, 3, 4, or 5; and
   i, j, and l are each independently 2, 3, 4, or 5;
   provided that
      when i is 4 and R²³ is —SR^{a}, or j is 4 and R³⁸ is —SR^{a}, or l is 4 and R⁵³ is —SR^{a}, then the R^{a} of the —SR^{a} is a substituted or unsubstituted C₁-C₆ alkyl group;
      when J is —NH₂, b and d are 0, a, c, e, f are 1, then R¹⁰³ is

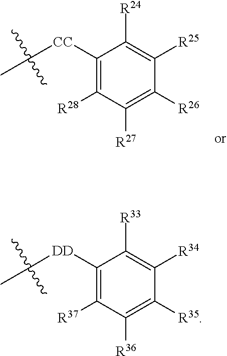

In some embodiments of peptides of Formula I,
R¹, R², R³, R⁴, and R⁵ are each independently a hydrogen or substituted or unsubstituted C₁-C₆ alkyl group;
R⁶ and R⁷ at each occurrence are independently a hydrogen or methyl group;
R⁸, R¹², R¹⁸, R²², R²⁴, R²⁸, R³³, R³⁷, R³⁹, R⁴³, R⁴⁸, R⁵², R⁵⁴, R⁵⁸, R⁶⁰, and R⁶⁴ are each independently a hydrogen or methyl group;
R¹⁰, R²⁰, R²⁶, R³⁵, R⁴¹, R⁵⁰, R⁵⁶, and R⁶² are each independently a hydrogen or —OR^{a};
R⁹, R¹¹, R¹⁹, R²¹, R²⁵, R²⁷, R³⁴, R³⁶, R⁴⁰, R⁴², R⁴⁹, R⁴⁰, R⁴², R⁴⁹, R⁵¹, R⁵⁵, R⁵⁷, R⁶¹, R⁶³, R⁶⁵, R⁶⁶, R⁶⁷, R⁶⁸, R⁶⁹, R⁷⁰, R⁷¹, R⁷², and R⁷³ are each a hydrogen;
R¹⁷, R²³, R³⁸, R⁵³, and R⁵⁹ are each independently a hydrogen, —OH, —SH, —SCH₃, —NH₂, —NHR^{b}, —CO₂H, —(CO)NH₂, —NH(CO)H, or —NH-dansyl group;
AA, BB, CC, DD, EE, FF, GG, and HH are each independently absent or —CH₂—;
R^{a} at each occurrence is independently a hydrogen or a substituted or unsubstituted C₁-C₆ alkyl group;
R^{b} at each occurrence is independently an ethylene-NH-dansyl or ethylene-NH-anthraniloyl group.

In some embodiments of Formula I,

A is

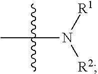

J is

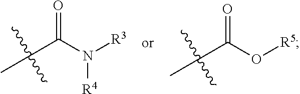

B, C, D, E, and G are each independently

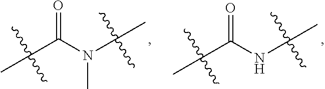

or absent;
with the proviso when f is 0, G is

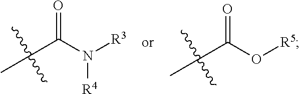

when e and f are 0, E is

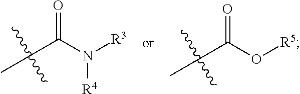

when d, e, and f are 0, D is

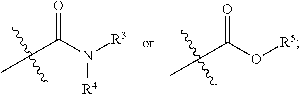

and
when c, d, e, and f are 0, C is

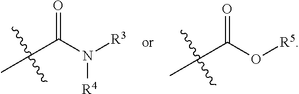

In another embodiment of Formula I,

A is

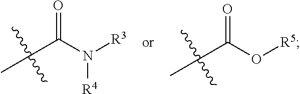

J is

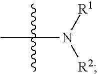

B, C, D, E, and G are each independently

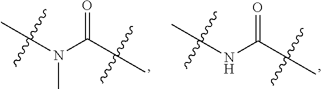

or absent;
with the proviso when f is 0, G is

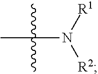

when e and f are 0, E is

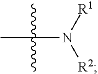

when d, e, and f are 0, D is

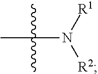

and
when c, d, e, and f are 0, C is

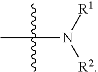

In some embodiments of Formula I, at least one of R¹⁰¹, R¹⁰², R¹⁰⁴, R¹⁰⁵, and R¹⁰⁶ is a basic group, as defined above, and at least one of R¹⁰¹, R¹⁰³, R¹⁰⁴, R¹⁰⁵, and R¹⁰⁶ is a neutral group as defined above. In some such embodiments, the neutral group is an aromatic, heterocyclic or cycloalkyl group as defined above. In some embodiments of Formula I, the peptide contains at least one arginine, such as, but not limited to D-arginine, and at least one 2′,6′-dimethyltyrosine, tyrosine, or phenylalanine. In some embodiments of Formula I, R¹⁰¹ is an alkylguanidinium group.

In some embodiments, the peptide of the present technology is selected from the peptides shown in Tables A or B.

TABLE A
Tyr-D-Arg-Phe-Lys-NH₂
D-Arg-Dmt-Lys-Phe-NH₂
D-Arg-Dmt-Phe-Lys-NH₂
D-Arg-Phe-Lys-Dmt-NH₂
D-Arg-Phe-Dmt-Lys-NH₂
D-Arg-Lys-Dmt-Phe-NH₂
D-Arg-Lys-Phe-Dmt-NH₂
D-Arg-Dmt-Lys-Phe-Cys-NH₂
Phe-Lys-Dmt-D-Arg-NH₂
Phe-Lys-D-Arg-Dmt-NH₂
Phe-D-Arg-Phe-Lys-NH₂
Phe-D-Arg-Phe-Lys-Cys-NH₂
Phe-D-Arg-Phe-Lys-Ser-Cys-NH₂
Phe-D-Arg-Phe-Lys-Gly-Cys-NH₂
Phe-D-Arg-Dmt-Lys-NH₂
Phe-D-Arg-Dmt-Lys-Cys-NH₂
Phe-D-Arg-Dmt-Lys-Ser-Cys-NH₂
Phe-D-Arg-Dmt-Lys-Gly-Cys-NH₂
Phe-D-Arg-Lys-Dmt-NH₂
Phe-Dmt-D-Arg-Lys-NH₂
Phe-Dmt-Lys-D-Arg-NH₂
Lys-Phe-D-Arg-Dmt-NH₂
Lys-Phe-Dmt-D-Arg-NH₂
Lys-Dmt-D-Arg-Phe-NH₂
Lys-Dmt-Phe-D-Arg-NH₂
Lys-D-Arg-Phe-Dmt-NH₂
Lys-D-Arg-Dmt-Phe-NH₂
D-Arg-Dmt-D-Arg-Phe-NH₂
D-Arg-Dmt-D-Arg-Dmt-NH₂
D-Arg-Dmt-D-Arg-Tyr-NH₂
D-Arg-Dmt-D-Arg-Trp-NH₂
Trp-D-Arg-Tyr-Lys-NH₂
Trp-D-Arg-Trp-Lys-NH₂
Trp-D-Arg-Dmt-Lys-NH₂
D-Arg-Trp-Lys-Phe-NH₂
D-Arg-Trp-Phe-Lys-NH₂
D-Arg-Trp-Lys-Dmt-NH₂
D-Arg-Trp-Dmt-Lys-NH₂
D-Arg-Lys-Trp-Phe-NH₂
D-Arg-Lys-Trp-Dmt-NH₂
Cha-D-Arg-Phe-Lys-NH₂
Ala-D-Arg-Phe-Lys-NH₂
2′,6′-Dmp-D-Arg-2′,6′-Dmt-Lys-NH₂
2′,6′-Dmp-D-Arg-Phe-Lys-NH₂
2′,6′-Dmt-D-Arg-Phe-Orn-NH₂
2′,6′-Dmt-D-Arg-Phe-Ahp-NH₂
2′,6′-Dmt-D-Arg-Phe-Lys-NH₂
2′,6′-Dmt-D-Cit-Phe-Lys-NH₂
D-Arg-2′,6′-Dmt-Lys-Phe-NH₂
D-Tyr-Trp-Lys-NH₂
Lys-D-Arg-Tyr-NH₂
Met-Tyr-D-Arg-Phe-Arg-NH₂
Met-Tyr-D-Lys-Phe-Arg
Phe-Arg-D-His-Asp
Phe-D-Arg-2′,6′-Dmt-Lys-NH₂
Phe-D-Arg-His
Trp-D-Lys-Tyr-Arg-NH₂
Tyr-D-Arg-Phe-Lys-Glu-NH₂
Tyr-His-D-Gly-Met
D-Arg-Tyr-Lys-Phe-NH₂
D-Arg-D-Dmt-Lys-Phe-NH₂
D-Arg-Dmt-D-Lys-Phe-NH₂
D-Arg-Dmt-Lys-D-Phe-NH₂
D-Arg-D-Dmt-D-Lys-D-Phe-NH₂
Phe-D-Arg-D-Phe-Lys-NH₂
Phe-D-Arg-Phe-D-Lys-NH₂
D-Phe-D-Arg-D-Phe-D-Lys-NH₂
Lys-D-Phe-Arg-Dmt-NH₂
D-Arg-Arg-Dmt-Phe-NH₂
Dmt-D-Phe-Arg-Lys-NH₂
Phe-D-Dmt-Arg-Lys-NH₂
D-Arg-Dmt-Lys-NH₂
Arg-D-Dmt-Lys-NH₂
D-Arg-Dmt-Phe-NH₂
Arg-D-Dmt-Arg-NH₂
Dmt-D-Arg-NH₂
D-Arg-Dmt-NH₂
D-Dmt-Arg-NH₂
Arg-D-Dmt-NH₂
D-Arg-D-Dmt-NH₂
D-Arg-D-Tyr-Lys-Phe-NH₂
D-Arg-Tyr-D-Lys-Phe-NH₂
D-Arg-Tyr-Lys-D-Phe-NH₂
D-Arg-D-Tyr-D-Lys-D-Phe-NH₂
Lys-D-Phe-Arg-Tyr-NH₂
D-Arg-Arg-Tyr-Phe-NH₂
Tyr-D-Phe-Arg-Lys-NH₂
Phe-D-Tyr-Arg-Lys-NH₂
D-Arg-Tyr-Lys-NH₂
Arg-D-Tyr-Lys-NH₂
D-Arg-Tyr-Phe-NH₂
Arg-D-Tyr-Arg-NH₂
Tyr-D-Arg-NH₂
D-Arg-Tyr-NH₂
D-Tyr-Arg-NH₂
Arg-D-Tyr-NH₂
D-Arg-D-Tyr-NH₂
Dmt-Lys-Phe-NH₂
Lys-Dmt-D-Arg-NH₂
Phe-Lys-Dmt-NH₂
D-Arg-Phe-Lys-NH₂
D-Arg-Cha-Lys-NH₂
D-Arg-Trp-Lys-NH₂
Dmt-Lys-D-Phe-NH₂
Dmt-Lys-NH₂
Lys-Phe-NH₂
D-Arg-Cha-Lys-Cha-NH₂
D-Nle-Dmt-Ahp-Phe-NH₂
D-Nle-Cha-Ahp-Cha-NH₂
D-Arg-Dmt-D-Lys-NH₂
D-Arg-Dmt-D-Lys-Phe-NH₂
Lys-Trp-D-Arg-NH₂
H-Lys-D-Phe-Arg-Dmt-NH₂
H-D-Arg-Lys-Dmt-Phe-NH₂
H-D-Arg-Lys-Phe-Dmt-NH₂
H-D-Arg-Arg-Dmt-Phe-NH₂
H-D-Arg-Dmt-Phe-Lys-NH₂
H-D-Arg-Phe-Dmt-Lys-NH₂
H-Dmt-D-Phe-Arg-Lys-NH₂
H-Phe-D-Dmt-Arg-Lys-NH₂
H-D-Arg-Dmt-Lys-NH₂
H-D-Arg-Dmt-D-Lys-D-Phe-NH₂
H-D-Arg-D-Dmt-Lys-Phe-NH₂
H-D-Arg-Dmt-Phe-NH₂
H-Dmt-D-Arg-NH₂
H-Phe-D-Arg-D-Phe-Lys-NH₂
H-Phe-D-Arg-Phe-D-Lys-NH₂
H-D-Phe-D-Arg-D-Phe-D-Lys-NH₂
H-D-Arg-D-Dmt-D-Lys-D-Phe-NH₂
H-D-Arg-Cha-Lys-NH₂
H-D-Arg-Cha-Lys-Cha-NH₂
H-Arg-D-Dmt-Lys-NH₂
H-Arg-D-Dmt-Arg-NH₂
H-D-Dmt-Arg-NH₂
H-Arg-D-Dmt-NH₂
H-D-Arg-D-Dmt-NH₂
Arg-Arg-Dmt-Phe
Arg-Cha-Lys
Arg-Dmt
Arg-Dmt-Arg
Arg-Dmt-Lys
Arg-Dmt-Lys-Phe
Arg-Dmt-Lys-Phe-Cys
Arg-Dmt-Phe
Arg-Dmt-Phe-Lys
Arg-Lys-Dmt-Phe
Arg-Lys-Phe-Dmt
Arg-Phe-Dmt-Lys
Arg-Phe-Lys
Arg-Trp-Lys
Arg-Tyr-Lys
Arg-Tyr-Lys-Phe
D-Arg-D-Dmt-D-Lys-L-Phe-NH₂
D-Arg-D-Dmt-L-Lys-D-Phe-NH₂
D-Arg-D-Dmt-L-Lys-L-Phe-NH₂
D-Arg-Dmt-D-Lys-NH₂
D-Arg-Dmt-Lys-NH₂
D-Arg-Dmt-Lys-Phe-Cys
D-Arg-L-Dmt-D-Lys-D-Phe-NH₂
D-Arg-L-Dmt-D-Lys-L-Phe-NH₂
D-Arg-L-Dmt-L-Lys-D-Phe-NH₂
Dmt-Arg
Dmt-Lys
Dmt-Lys-Phe
Dmt-Phe-Arg-Lys
H-Arg-D-Dmt-Lys-Phe-NH₂
H-Arg-Dmt-Lys-Phe-NH₂
H-D-Arg-2,6-dichloro-L-tyrosine-L-Lys-L-Phe-NH₂
H-D-Arg-2,6-dichlorotyrosine-Lys-Phe-NH₂
H-D-Arg-2,6-difluoro-L-tyrosine-L-Lys-L-Phe-NH₂
H-D-Arg-2,6-difluorotyrosine-Lys-Phe-NH₂
H-D-Arg-2,6-dimethyl-L-phenylalanine-L-Lys-L-
Phe-NH₂
H-D-Arg-2,6-dimethylphenylalanine-Lys-Phe-NH₂
H-D-Arg-4-methoxy-2,6-dimethyl-L-phenylalanine-
L-Lys-L-Phe-NH₂
H-D-Arg-4-methoxy-2,6-dimethylphenylalanine-Lys-
Phe-NH₂
H-D-Arg-Dmt-Lys-2,6-dimethylphenylalanine-NH₂
H-D-Arg-Dmt-Lys-3-hydroxyphenylalanine-NH₂
H-D-Arg-Dmt-N6-acetyllysine-Phe-NH₂
H-D-Arg-D-Phe-L-Lys-L-Phe-NH₂
H-D-Arg-D-Trp-L-Lys-L-Phe-NH₂
H-D-Arg-D-Tyr-L-Lys-L-Phe-N_{H2}
H-D-Arg-L-Dmt-L-Lys-2,6-dimethyl-L-
phenylalanine-NH₂
H-D-Arg-L-Dmt-L-Lys-3-hydroxy-L-phenylalanine-NH₂
H-D-Arg-L-Dmt-L-Lys-D-Dmt-NH₂
H-D-Arg-L-Dmt-L-Lys-D-Trp-NH₂
H-D-Arg-L-Dmt-L-Lys-D-Tyr-NH₂
H-D-Arg-L-Dmt-L-Lys-L-Dmt-NH₂
H-D-Arg-L-Dmt-L-Lys-L-Trp-NH₂
H-D-Arg-L-Dmt-L-Lys-L-Tyr-NH₂
H-D-Arg-L-Dmt-L-Phe-L-Lys-NH₂
H-D-Arg-L-Dmt-N6-acetyl-L-lysine-L-Phe-NH₂
H-D-Arg-L-Lys-L-Dmt-L-Phe-NH₂
H-D-Arg-L-Lys-L-Phe-L-Dmt-NH₂
H-D-Arg-L-Phe-L-Dmt-L-Lys-NH₂
H-D-Arg-L-Phe-L-Lys-L-Dmt-NH₂
H-D-Arg-L-Phe-L-Lys-L-Phe-NH₂
H-D-Arg-L-Trp-L-Lys-L-Phe-NH₂
H-D-Arg-L-Tyr-L-Lys-L-Phe-NH₂
H-D-Arg-Phe-Lys-Dmt-NH₂
H-D-Arg-Tyr-Lys-Phe-NH₂
H-D-His-L-Dmt-L-Lys-L-Phe-NH,
H-D-Lys-L-Dmt-L-Lys-L-Phe-NH₂
H-Dmt-D-Arg-Lys-Phe-NH₂
H-Dmt-D-Arg-Phe-Lys-NH₂
H-Dmt-Lys-D-Arg-Phe-NH₂
H-Dmt-Lys-Phe-D-Arg-NH₂
H-Dmt-Phe-D-Arg-Lys-NH₂
H-Dmt-Phe-Lys-D-Arg-NH₂
H-L-Dmt-D-Arg-L-Lys-L-Phe-NH₂
H-L-Dmt-D-Arg-L-Phe-L-Lys-NH₂
H-L-Dmt-L-Lys-D-Arg-L-Phe-NH₂
H-L-Dmt-L-Lys-L-Phe-D-Arg-NH₂
H-L-Dmt-L-Phe-D-Arg-L-Lys-NH₂
H-L-Dmt-L-Phe-L-Lys-D-Arg-NH₂
H-L-His-L-Dmt-L-Lys-L-Phe-NH₂
H-L-Lys-D-Arg-L-Dmt-L-Phe-NH₂
H-L-Lys-D-Arg-L-Phe-L-Dmt-NH₂
H-L-Lys-L-Dmt-D-Arg-L-Phe-NH₂
H-L-Lys-L-Dmt-L-Lys-L-Phe-NH,
H-L-Lys-L-Dmt-L-Phe-D-Arg-NH₂
H-L-Lys-L-Phe-D-Arg-L-Dmt-NH₂
H-L-Lys-L-Phe-L-Dmt-D-Arg-NH₂
H-L-Phe-D-Arg-L-Dmt-L-Lys-NH₂
H-L-Phe-D-Arg-L-Lys-L-Dmt-NH₂
H-L-Phe-L-Dmt-D-Arg-L-Lys-NH₂
H-L-Phe-L-Dmt-L-Lys-D-Arg-NH₂
H-L-Phe-L-Lys-D-Arg-L-Dmt-NH₂
H-L-Phe-L-Lys-L-Dmt-D-Arg-NH₂
H-Lys-D-Arg-Dmt-Phe-NH₂
H-Lys-D-Arg-Phe-Dmt-NH₂
H-Lys-Dmt-D-Arg-Phe-NH₂
H-Lys-Dmt-Phe-D-Arg-NH₂
H-Lys-Phe-D-Arg-Dmt-NH₂
H-Lys-Phe-Dmt-D-Arg-NH₂
H-Phe-Arg-Phe-Lys-NH₂
H-Phe-D-Arg-Dmt-Lys-NH₂
H-Phe-D-Arg-Lys-Dmt-NH₂
H-Phe-Dmt-D-Arg-Lys-NH₂
H-Phe-Dmt-Lys-D-Arg-NH₂
H-Phe-Lys-D-Arg-Dmt-NH₂
H-Phe-Lys-Dmt-D-Arg-NH₂
L-Arg-D-Dmt-D-Lys-D-Phe-NH₂
L-Arg-D-Dmt-D-Lys-L-Phe-NH₂
L-Arg-D-Dmt-L-Lys-D-Phe-NH₂
L-Arg-D-Dmt-L-Lys-L-Phe-NH₂
L-Arg-L-Dmt-D-Lys-D-Phe-NH₂
L-Arg-L-Dmt-D-Lys-L-Phe-NH₂
L-Arg-L-Dmt-L-Lys-D-Phe-NH₂
L-Arg-L-Dmt-L-Lys-L-Phe-NH₂
Lys-Dmt-Arg
Lys-Phe
Lys-Phe-Arg-Dmt
Lys-Trp-Arg
Phe-Arg-Dmt-Lys
Phe-Arg-Phe-Lys
Phe-Dmt-Arg-Lys
Phe-Lys-Dmt
Arg-Dmt-Lys-Phe-NH₂
Phe-Dmt-Arg-Lys-NH₂
Phe-Lys-Dmt-Arg-NH₂
Dmt-Arg-Lys-Phe-NH₂
Lys-Dmt-Arg-Phe-NH₂
Phe-Dmt-Lys-Arg-NH₂
Arg-Lys-Dmt-Phe-NH₂
Arg-Dmt-Phe-Lys-NH₂
D-Arg-Dmt-Lys-Phe-NH₂
Dmt-D-Arg-Phe-Lys-NH₂
H-Phe-D-Arg Phe-Lys-Cys-NH₂
D-Arg-Dmt-Lys-Trp-NH₂
D-Arg-Trp-Lys-Trp-NH₂
H-D-Arg-Dmt-Lys-Phe(NMe)-NH₂
H-D-Arg-Dmt-Lys(N^{α}Me)-Phe(NMe)-NH₂
H-D-Arg(N^{α}Me)-Dmt(NMe)-Lys(N^{α}Me)-Phe(NMe)-NH₂
D-Arg-(N^{α}Me)-Dmt-Lys-Phe-NH₂
H-Phe-D-Arg-Phe-Lys-Cys-NH₂
D-Arg-Dmt-Lys-Phe-Ser-Cys-NH₂
D-Arg-Dmt-Lys-Phe-Gly-Cys-NH₂
Gly-D-Phe-Lys-His-D-Arg-Tyr-NH₂
D-Arg-Dmt-Lys-Phe-Met-NH₂
D-Arg-Dmt-Lys-Phe-Lys-Trp-NH₂
D-Arg-Dmt-Lys-Dmt-Lys-Trp-NH₂
D-Arg-Dmt-Lys-Phe-Lys-Met-NH₂
D-Arg-Dmt-Lys-Dmt-Lys-Met-NH₂
H-D-Arg-Dmt-Lys-OH
H-D-Arg-Dmt-OH
H-D-Arg-Dmt-Lys-Phe-OH

TABLE B

| Amino Acid | Amino Acid | Amino Acid | Amino Acid | C-Terminal |
|---|---|---|---|---|
| Position 1 | Position 2 | Position 3 | Position 4 | Modification |
| Tyr | D-Arg | Phe | Orn | NH₂ |
| Tyr | D-Arg | Phe | Dab | NH₂ |
| Tyr | D-Arg | Phe | Dap | NH₂ |
| 2′6′Dmt | D-Arg | Phe | Lys-NH(CH₂)₂—NH-dns | NH₂ |
| 2′6′Dmt | D-Arg | Phe | Lys-NH(CH₂)₂—NH-atn | NH₂ |
| 2′6′Dmt | D-Arg | Phe | dnsLys | NH₂ |
| 2′6′Dmt | D-Cit | Phe | Ahp | NH₂ |
| 2′6′Dmt | D-Arg | Phe | Dab | NH₂ |
| 2′6′Dmt | D-Arg | Phe | Dap | NH₂ |
| 3′5′Dmt | D-Arg | Phe | Lys | NH₂ |
| 3′5′Dmt | D-Arg | Phe | Orn | NH₂ |
| 3′5′Dmt | D-Arg | Phe | Dab | NH₂ |
| 3′5′Dmt | D-Arg | Phe | Dap | NH₂ |
| Tyr | D-Arg | Tyr | Lys | NH₂ |
| Tyr | D-Arg | Tyr | Orn | NH₂ |
| Tyr | D-Arg | Tyr | Dab | NH₂ |
| Tyr | D-Arg | Tyr | Dap | NH₂ |
| 2′6′Dmt | D-Arg | Tyr | Lys | NH₂ |
| 2′6′Dmt | D-Arg | Tyr | Orn | NH₂ |
| 2′6′Dmt | D-Arg | Tyr | Dab | NH₂ |
| 2′6′Dmt | D-Arg | Tyr | Dap | NH₂ |
| 2′6′Dmt | D-Arg | 2′6′Dmt | Lys | NH₂ |
| 2′6′Dmt | D-Arg | 2′6′Dmt | Orn | NH₂ |
| 2′6′Dmt | D-Arg | 2′6′Dmt | Dab | NH₂ |
| 2′6′Dmt | D-Arg | 2′6′Dmt | Dap | NH₂ |
| 3′5′Dmt | D-Arg | 3′5′Dmt | Arg | NH₂ |
| 3′5′Dmt | D-Arg | 3′5′Dmt | Lys | NH₂ |
| 3′5′Dmt | D-Arg | 3′5′Dmt | Orn | NH₂ |
| 3′5′Dmt | D-Arg | 3′5′Dmt | Dab | NH₂ |
| Tyr | D-Lys | Phe | Dap | NH₂ |
| Tyr | D-Lys | Phe | Arg | NH₂ |
| Tyr | D-Lys | Phe | Lys | NH₂ |
| Tyr | D-Lys | Phe | Orn | NH₂ |
| 2′6′Dmt | D-Lys | Phe | Dab | NH₂ |
| 2′6′Dmt | D-Lys | Phe | Dap | NH₂ |
| 2′6′Dmt | D-Lys | Phe | Arg | NH₂ |
| 2′6′Dmt | D-Lys | Phe | Lys | NH₂ |
| 3′5′Dmt | D-Lys | Phe | Orn | NH₂ |
| 3′5′Dmt | D-Lys | Phe | Dab | NH₂ |
| 3′5′Dmt | D-Lys | Phe | Dap | NH₂ |
| 3′5′Dmt | D-Lys | Phe | Arg | NH₂ |
| Tyr | D-Lys | Tyr | Lys | NH₂ |
| Tyr | D-Lys | Tyr | Orn | NH₂ |
| Tyr | D-Lys | Tyr | Dab | NH₂ |
| Tyr | D-Lys | Tyr | Dap | NH₂ |
| 2′6′Dmt | D-Lys | Tyr | Lys | NH₂ |
| 2′6′Dmt | D-Lys | Tyr | Orn | NH₂ |
| 2′6′Dmt | D-Lys | Tyr | Dab | NH₂ |
| 2′6′Dmt | D-Lys | Tyr | Dap | NH₂ |
| 2′6′Dmt | D-Lys | 2′6′Dmt | Lys | NH₂ |
| 2′6′Dmt | D-Lys | 2′6′Dmt | Orn | NH₂ |
| 2′6′Dmt | D-Lys | 2′6′Dmt | Dab | NH₂ |
| 2′6′Dmt | D-Lys | 2′6′Dmt | Dap | NH₂ |
| 3′5′Dmt | D-Lys | 3′5′Dmt | Lys | NH₂ |
| 3′5′Dmt | D-Lys | 3′5′Dmt | Orn | NH₂ |
| 3′5′Dmt | D-Lys | 3′5′Dmt | Dab | NH₂ |
| 3′5′Dmt | D-Lys | 3′5′Dmt | Dap | NH₂ |
| Tyr | D-Lys | Phe | Arg | NH₂ |
| Tyr | D-Orn | Phe | Arg | NH₂ |
| Tyr | D-Dab | Phe | Arg | NH₂ |
| Tyr | D-Dap | Phe | Arg | NH₂ |
| 2′6′Dmt | D-Arg | Phe | Arg | NH₂ |
| 2′6′Dmt | D-Lys | Phe | Arg | NH₂ |
| 2′6′Dmt | D-Orn | Phe | Arg | NH₂ |
| 2′6′Dmt | D-Dab | Phe | Arg | NH₂ |
| 3′5′Dmt | D-Dap | Phe | Arg | NH₂ |
| 3′5′Dmt | D-Arg | Phe | Arg | NH₂ |
| 3′5′Dmt | D-Lys | Phe | Arg | NH₂ |
| 3′5′Dmt | D-Orn | Phe | Arg | NH₂ |
| Tyr | D-Lys | Tyr | Arg | NH₂ |
| Tyr | D-Orn | Tyr | Arg | NH₂ |
| Tyr | D-Dab | Tyr | Arg | NH₂ |
| Tyr | D-Dap | Tyr | Arg | NH₂ |
| 2′6′Dmt | D-Arg | 2′6′Dmt | Arg | NH₂ |
| 2′6′Dmt | D-Lys | 2′6′Dmt | Arg | NH₂ |
| 2′6′Dmt | D-Orn | 2′6′Dmt | Arg | NH₂ |
| 2′6′Dmt | D-Dab | 2′6′Dmt | Arg | NH₂ |
| 3′5′Dmt | D-Dap | 3′5′Dmt | Arg | NH₂ |
| 3′5′Dmt | D-Arg | 3′5′Dmt | Arg | NH₂ |
| 3′5′Dmt | D-Lys | 3′5′Dmt | Arg | NH₂ |
| 3′5′Dmt | D-Orn | 3′5′Dmt | Arg | NH₂ |
| Mmt | D-Arg | Phe | Lys | NH₂ |
| Mmt | D-Arg | Phe | Orn | NH₂ |
| Mmt | D-Arg | Phe | Dab | NH₂ |
| Mmt | D-Arg | Phe | Dap | NH₂ |
| Tmt | D-Arg | Phe | Lys | NH₂ |
| Tmt | D-Arg | Phe | Orn | NH₂ |
| Tmt | D-Arg | Phe | Dab | NH₂ |
| Tmt | D-Arg | Phe | Dap | NH₂ |
| Hmt | D-Arg | Phe | Lys | NH₂ |
| Hmt | D-Arg | Phe | Orn | NH₂ |
| Hmt | D-Arg | Phe | Dab | NH₂ |
| Hmt | D-Arg | Phe | Dap | NH₂ |
| Mmt | D-Lys | Phe | Lys | NH₂ |
| Mmt | D-Lys | Phe | Orn | NH₂ |
| Mmt | D-Lys | Phe | Dab | NH₂ |
| Mmt | D-Lys | Phe | Dap | NH₂ |
| Mmt | D-Lys | Phe | Arg | NH₂ |
| Tmt | D-Lys | Phe | Lys | NH₂ |
| Tmt | D-Lys | Phe | Orn | NH₂ |
| Tmt | D-Lys | Phe | Dab | NH₂ |
| Tmt | D-Lys | Phe | Dap | NH₂ |
| Tmt | D-Lys | Phe | Arg | NH₂ |
| Hmt | D-Lys | Phe | Lys | NH₂ |
| Hmt | D-Lys | Phe | Orn | NH₂ |
| Hmt | D-Lys | Phe | Dab | NH₂ |
| Hmt | D-Lys | Phe | Dap | NH₂ |
| Hmt | D-Lys | Phe | Arg | NH₂ |
| Mmt | D-Lys | Phe | Arg | NH₂ |
| Mmt | D-Orn | Phe | Arg | NH₂ |
| Mmt | D-Dab | Phe | Arg | NH₂ |
| Mmt | D-Dap | Phe | Arg | NH₂ |
| Mmt | D-Arg | Phe | Arg | NH₂ |
| Tmt | D-Lys | Phe | Arg | NH₂ |
| Tmt | D-Orn | Phe | Arg | NH₂ |
| Tmt | D-Dab | Phe | Arg | NH₂ |
| Tmt | D-Dap | Phe | Arg | NH₂ |
| Tmt | D-Arg | Phe | Arg | NH₂ |
| Hmt | D-Lys | Phe | Arg | NH₂ |
| Hmt | D-Orn | Phe | Arg | NH₂ |
| Hmt | D-Dab | Phe | Arg | NH₂ |
| Hmt | D-Dap | Phe | Arg | NH₂ |
| Hmt | D-Arg | Phe | Arg | NH₂ |
| Trp | D-Arg | Phe | Lys | NH₂ |

2′-methyltyrosine (Mmt); Dimethyltyrosine (Dmt); 2′,6′-dimethyltyrosine (2′6′-Dmt); 3′,5′-dimethyltyrosine (3′S′Dmt); N,2′,6′-trimethyltyrosine (Tmt); 2′-hydroxy-6′-methyltyrosine (Hmt); 2′-methylphenylalanine (Mmp); dimethylphenylalanine (Dmp) 2′,6′-dimethylphenylalanine (2′,6′-Dmp); N,2′,6′-trimethylphenylalanine (Tmp); 2′-hydroxy-6′-methylphenylalanine (Hmp); cyclohexylalanine (Cha); diaminobutyric (Dab); diaminopropionic acid (Dap); β-dansyl-L-α,β-diaminopropionic acid (dnsDap); β-anthraniloyl-L-αβ-diaminopropionic acid (atnDap); biotin (bio); norleucine (Nle); 2-aminohepantoic acid (Ahp); β-(6′-dimethylamino-2′-naphthoyl)alanine (Ald); Sarcosine (Sar)

In another embodiment, the peptide is defined by Formula II:

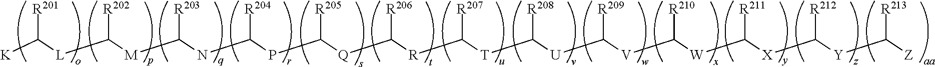

wherein:

one of K and Z is

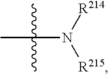

and the other of K and Z is

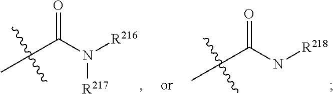

L, M, N, P, Q, R, T, U, V, W, X, and Y are each

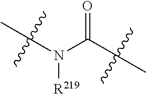

or L, M, N, P, Q, R, T, U, V, W, X, and Y are each

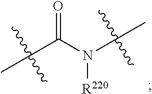

with the proviso that when
   aa is 0 and Z is not a terminal group, the terminal group is one of L, M, N, P, Q, R, T, U, V, W, X, or Y, such that one of K and the terminal group is

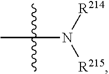

and the other of K and the terminal group is selected from

R²⁰¹ is

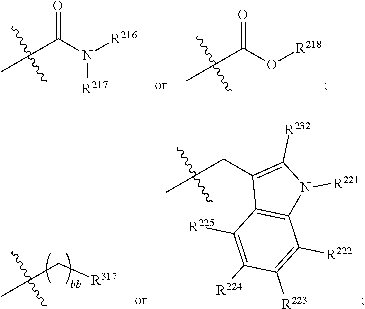

R²⁰² is

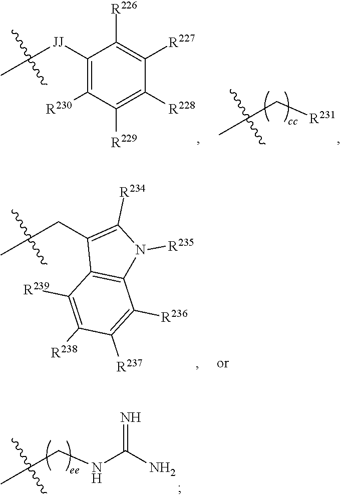

R²⁰³ is

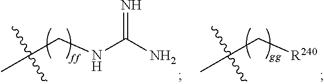

or hydrogen;

R²⁰⁴ is

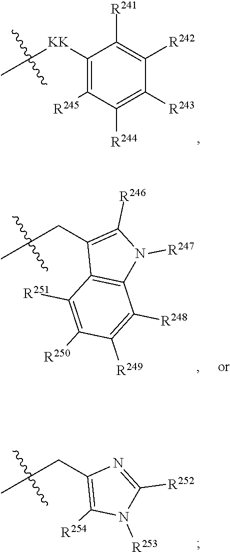

R²⁰⁵ is

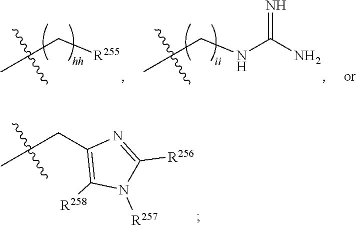

R²⁰⁶ is

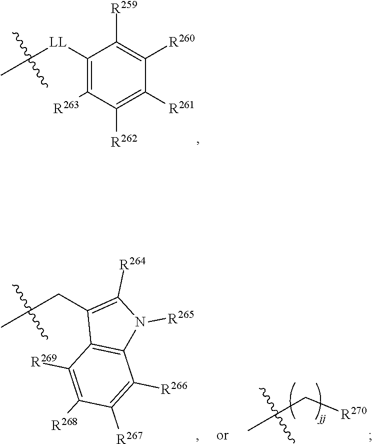

R²⁰⁷ is

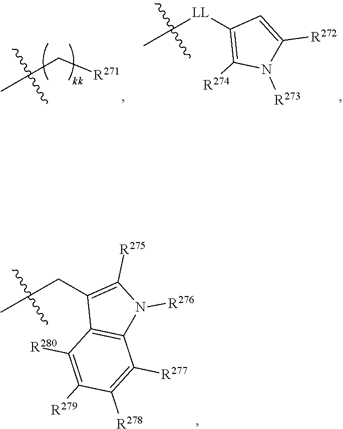

or hydrogen;

R²⁰⁸ is

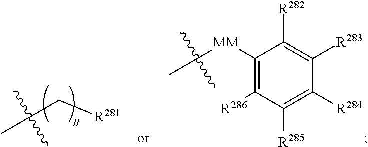

R²⁰⁹ is

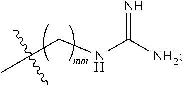

R²¹⁰ is

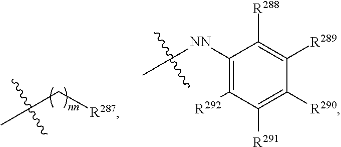

or hydrogen;

R²¹¹ is

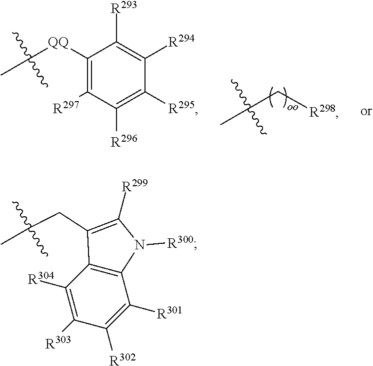

R²¹² is

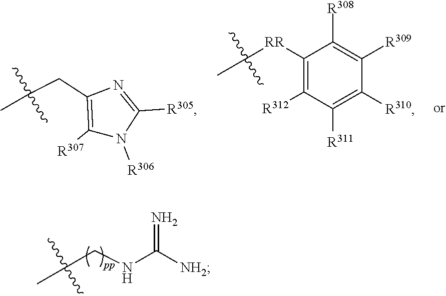

R²¹³ is

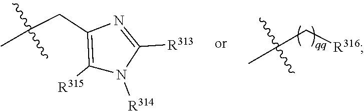

wherein
   R²¹⁴, R²¹⁵, R²¹⁶, R²¹⁷, and R²¹⁸ are each independently a hydrogen or substituted or unsubstituted C₁-C₆ alkyl, C₂-C₆ alkenyl, C₂-C₆ alkynyl, saturated or unsaturated cycloalkyl, cycloalkylalkyl, aryl, aralkyl, 5- or 6-membered saturated or unsaturated heterocylyl, heteroaryl, or amino protecting group; or R²¹⁴ and R²¹⁵together form a 3, 4, 5, 6, 7, or 8 membered substituted or unsubstituted heterocycyl ring;
   R²¹⁹ and R²²⁰ are, at each occurrence, independently a hydrogen or substituted or unsubstituted C₁-C₆ alkyl group;
   R²²², R²²³, R²²⁴, R²²⁵, R²²⁶, R²²⁷, R²²⁸, R²²⁹, R²³⁰, R²³², R²³⁴, R²³⁶, R²³⁷, R²³⁸, R²³⁹, R²⁴¹, R²⁴², R²⁴³, R²⁴⁴, R²⁴⁵, R²⁴⁶, R²⁴⁸, R²⁴⁹, R²⁵⁰, R²⁵¹, R²⁵², R²⁵⁴, R²⁵⁶, R²⁵⁸, R²⁵⁹, R²⁶⁰, R²⁶¹, R²⁶², R²⁶³, R²⁶⁴, R²⁶⁶, R²⁶⁷, R²⁶⁸, R²⁶⁹, R²⁷², R²⁷⁴, R²⁷⁵, R²⁷⁷, R²⁷⁸, R²⁷⁹, R²⁸⁰, R²⁸², R²⁸³, R²⁸⁴, R²⁸⁵, R²⁸⁶, R²⁸⁸, R²⁸⁹, R²⁹⁰, R²⁹¹, R²⁹², R²⁹³, R²⁹⁴, R²⁹⁵, R²⁹⁶, R²⁹⁷, R²⁹⁹, R³⁰¹, R³⁰², R³⁰³, R³⁰⁴, R³⁰⁵, R³⁰⁷, R³⁰⁸, R³⁰⁹, R³¹⁰, R³¹¹, R³¹², R³¹³, and R³¹⁵ are each independently a hydrogen, amino, amido, —NO₂, —CN, —OR', —SR', —NR^{c}R^{c}, —F, —Cl, —Br, —I, or a substituted or unsubstituted C₁-C₆ alkyl, C₁-C₆ alkoxy, —C(O)-alkyl, —C(O)-aryl, —C(O)-aralkyl, —C(O)₂R^{c}, C₁-C₄ alkylamino, C₁-C₄ dialkylamino, or perhaloalkyl group;
   R²²¹, R²³⁵, R²⁴⁷, R²⁵³, R²⁵⁷, R²⁶⁵, R²⁷³, R²⁷⁶, R³⁰⁰, R³⁰⁶, and R³¹⁴ are each independently a hydrogen or substituted or unsubstituted C₁-C₆ alkyl group;
   R²³¹, R²⁴⁰, R²⁵⁵, R²⁷⁰, R²⁷¹, R²⁸¹, R²⁸⁷, R²⁹⁸, R³¹⁶, and R³¹⁷ are each independently a hydrogen, —OR^{c}, —SR^{c}, —NR^{c}R^{c}, —NR^{c}R^{d}, —CO₂R^{c}, —(CO)NR^{c}R^{c}, —NR^{c}(CO)R^{c}, —NR^{c}C(NH)NH₂, —NR^{c}-dansyl, or a substituted or unsubstituted alkyl, aryl, or aralkyl group;
   JJ, KK, LL, MM, NN, QQ, and RR are each independently absent, —NH(CO)—, or —CH₂—;
   R^{c} at each occurrence is independently a hydrogen or a substituted or unsubstituted C₁-C₆ alkyl group;
   R^{d} at each occurrence is independently a C₁-C₆ alkylene-NR^{c}-dansyl or C₁-C₆ alkylene-NR^{c}-anthraniloyl group;
   o, p, q, r, s, t, u, v, w, x, y, z, and aa are each independently 0 or 1, with the proviso that o+p+q+r+s+t +u+v+w+x+y+z+aa equals 6, 7, 8, 9, 10, or 11;
   cc is 0, 1, 2, 3, 4, or 5; and
   bb, cc, ee, ff, gg, hh, ii, jj, kk, ll, mm, nn, oo, pp, and qq are each independently 1, 2, 3, 4, or 5.

In some embodiments of peptides of Formula II,
R²¹⁴, R²¹⁵, R²¹⁶, R²¹⁷, and R²¹⁸ are each independently a hydrogen or substituted or unsubstituted C₁-C₆ alkyl group;
R²¹⁹ and R²²⁰ are, at each occurrence, independently a hydrogen or methyl group;
R²²², R²²³, R²²⁴, R²²⁵, R²²⁶, R²²⁷, R²²⁸, R²²⁹, R²³⁰, R²³², R²³⁴, R²³⁶, R²³⁷, R²³⁸, R²³⁹, R²⁴¹, R²⁴², R²⁴³, R²⁴⁴, R²⁴⁵, R²⁴⁶, R²⁴⁸, R²⁴⁹, R²⁵⁰, R²⁵¹, R²⁵², R²⁵⁴, R²⁵⁶, R²⁵⁸, R²⁵⁹, R²⁶⁰, R²⁶¹, R²⁶², R²⁶³, R²⁶⁴, R²⁶⁶, R²⁶⁷, R²⁶⁸, R²⁶⁹, R²⁷², R²⁷⁴, R²⁷⁵, R²⁷⁷, R²⁷⁸, R²⁷⁹, R²⁸⁰, R²⁸², R²⁸³, R²⁸⁴, R²⁸⁵, R²⁸⁶, R²⁸⁸, R²⁸⁹, R²⁹⁰, R²⁹¹, R²⁹², R²⁹³, R²⁹⁴, R²⁹⁵, R²⁹⁶, R²⁹⁷, R²⁹⁹, R³⁰¹, R³⁰², R³⁰³, R³⁰⁴, R³⁰⁵, R³⁰⁷, R³⁰⁸, R³⁰⁹, R³¹⁰, R³¹¹, R³¹², R³¹³, and R³¹⁵ are each independently a hydrogen, methyl, or —OR^{c} group;
R²²¹, R²³⁵, R²⁴⁷, R²⁵³, R²⁵⁷, R²⁶⁵, R²⁷³, R²⁷⁶, R³⁰⁰, R³⁰⁶, and R³¹⁴ are each independently a hydrogen or substituted or unsubstituted C₁-C₆ alkyl group;
R²³¹ is —(CO)NR^{c}R^{c}, —OR^{c}, or a C₁-C₆ alkyl group, optionally substituted with a hydroxyl or methyl group;
R²⁴⁰ and R²⁵⁵ are each independently —CO₂R^{c} or —NR^{c}R^{c};
R²⁷⁰ are each independently —CO₂R^{c};
R²⁸¹ is —SR^{c} or —NR^{c}R^{c};
R²⁸⁷ —(CO)NR^{c}R^{c} or —OR^{c};
R²⁹⁸ —NR^{c}R^{c}, —CO₂R^{c}, or —SR^{c};
R³¹⁶ is —NR^{c}R^{c};
R³¹⁷ is hydrogen or —NR^{c}R^{c};
JJ, KK, LL, MM, NN, QQ, and RR are each independently absent or —CH₂—;
R^{c} at each occurrence is independently a hydrogen or a substituted or unsubstituted C₁-C₆ alkyl group;
R^{d} at each occurrence is independently a C₁-C₆ alkylene-NR^{c}-dansyl or C₁-C₆ alkylene-NR^{c}-anthraniloyl group;
o, p, q, r, s, t, u, v, w, x, y, z, and aa are each independently 0 or 1, with the proviso that o+p+q+r+s+t+u+v+w+x+y+z+aa equals 6, 7, 8, 9, 1 0, or 11;
cc is 0, 1, 2, 3, 4, or 5; and
bb, cc, dd, ee, ff, gg, hh, ii, jj, kk, ll, mm, nn, oo, pp, and qq are each independently 1, 2, 3, 4, or 5.

In some embodiments of peptides of Formula II,
R²²¹, R²²², R²²³, R²²⁴, R²²⁵, R²²⁶, R²²⁷, R²²⁸, R²²⁹, R²³⁰, R²³², R²³⁴, R²³⁵, R²³⁶, R²³⁷, R²³⁸, R²³⁹, R²⁴², R²⁴⁴, R²⁴⁶, R²⁴⁷, R²⁴⁸, R²⁴⁹, R²⁵⁰, R²⁵¹, R²⁵², R²⁵³, R²⁵⁴, R²⁵⁶, R²⁵⁷, R²⁵⁸, R²⁵⁹, R²⁶⁰, R²⁶², R²⁶³, R²⁶⁴, R²⁶⁵, R²⁶⁶, R²⁶⁷, R²⁶⁸, R²⁶⁹, R²⁷², R²⁷³, R²⁷⁴, R²⁷⁵, R²⁷⁶, R²⁷⁷, R²⁷⁸, R²⁷⁹, R²⁸⁰, R²⁸², R²⁸³, R²⁸⁵, R²⁸⁶, R²⁸⁸, R²⁸⁹, R²⁹¹, R²⁹², R²⁹³, R²⁹⁴, R²⁹⁶, R²⁹⁷, R²⁹⁹, R³⁰⁰, R³⁰¹, R³⁰², R³⁰³, R³⁰⁴, R³⁰⁵, R³⁰⁶, R³⁰⁷, R³⁰⁸, R³⁰⁹, R³¹¹, R³¹², R³¹³, R³¹⁴, and R³¹⁵ are each hydrogen;
R²⁴¹ and R²⁴⁵ are each independently a hydrogen or methyl group;
R²⁴³, R²⁶¹, R²⁸⁴, R²⁹⁰, R²⁹⁵, R³¹⁰ are each independently a hydrogen or OH;
R²³¹ is —(CO)NH₂, an ethyl group substituted with a hydroxyl group, or an isopropyl group;
R²⁴⁰ and R²⁵⁵ are each independently —CO₂H or —NH₂;
R²⁷⁰ and R²⁷¹ are each independently —CO₂H;
R²⁸¹ is —SH or —NH₂;
R²⁸⁷ is —(CO)NH₂ or —OH;
R²⁹⁸ is —NH₂, —CO₂H, or —SH;
R³¹⁶ is —NH₂;
R³¹⁷ is hydrogen or —NH₂;
JJ, KK, LL, MM, NN, QQ, and RR are each independently —CH₂₋;
o, p, q, r, s, t, u, v, w, x, y, z, and aa are each independently 0 or 1, with the proviso that o+p+q+r+s+t+u+v+w+x+y+z+aa equals 6, 7, 8, 9, 10, or 11;
cc is 0, 1, 2, 3, 4, or 5; and
bb, cc, dd, ee, ff, gg, hh, ii, jj, kk, ll, mm, nn, oo, pp, and qq are each independently 1, 2, 3, 4, or 5.

In certain embodiments of Formula II,

K is

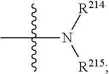

Z is

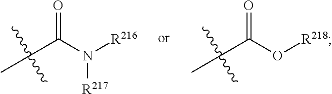

L, M, N, P, Q, R, T, U, V, W, X, and Y are each independently

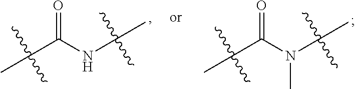

with the proviso that when
   aa is 0 and Z is not a terminal group, the terminal group is one of L, M, N, P, Q, R, T, U, V, W, X, or Y, such that one of L, M, N, P, Q, R, T, U, V, W, X, or Y, is

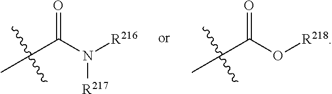

In another embodiment of Formula II,

K is

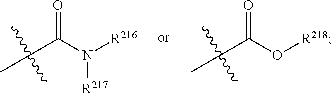

Z is

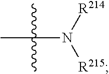

L, M, N, P, Q, R, T, U, V, W, X, and Y are each independently

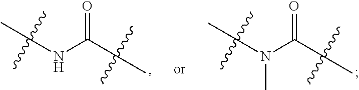

with the proviso that when aa is 0 and Z is not a terminal group, the terminal group is one of L, M, N, P, Q, R, T, U, V, W, X, or Y, such that one of L, M, N, P, Q, R, T, U, V, W, X, or Y, is

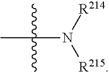

In some embodiments, the peptide of Formula II is selected from the peptides shown in Table C.

TABLE C
D-Arg-Dmt-Lys-Phe-Glu-Cys-Gly-NH₂
Phe-D-Arg-Phe-Lys-Glu-Cys-Gly-NH₂
Phe-D-Arg-Dmt-Lys-Glu-Cys-Gly-NH₂
Ala-D-Phe-D-Arg-Tyr-Lys-D-Trp-His-D-Tyr-Gly-Phe
Asp-D-Trp-Lys-Tyr-D-His-Phe-Arg-D-Gly-Lys-NH₂
D-His-Glu-Lys-Tyr-D-Phe-Arg
D-His-Lys-Tyr-D-Phe-Glu-D-Asp-D-Asp-D-His-D-Lys-
Arg-Trp-NH₂
Lys-D-Gln-Tyr-Arg-D-Phe-Trp-NH₂
Lys-Trp-D-Tyr-Arg-Asn-Phe-Tyr-D-His-NH₂
Phe-D-Arg-Lys-Trp-Tyr-D-Arg-His
Thr-Gly-Tyr-Arg-D-His-Phe-Trp-D-His-Lys
Trp-Lys-Phe-D-Asp-Arg-Tyr-D-His-Lys
Val-D-Lys-His-Tyr-D-Phe-Ser-Tyr-Arg-NH₂
Gly-D-Phe-Lys-Tyr-His-D-Arg-Tyr-NH₂
Asp-D-Trp-Lys-Tyr-D-His-Phe-Arg-D-Gly-Lys-NH₂
D-His-Lys-Tyr-D-Phe-Glu-D-Asp-D-His-D-Lys-Arg-
Trp-NH₂
H-Phe-D-Arg-Phe-Lys-Glu-Cys-Gly-NH₂
Phe-Arg-Phe-Lys-Glu-Cys-Gly
H-D-Arg-Dmt-Lys-Phe-Sar-Gly-Cys-NH₂

In another embodiment the peptide is defined by Formula III:

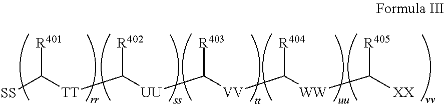

wherein:

one of SS and XX is

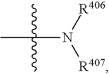

and the other is

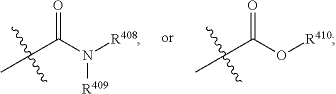

TT, UU, VV, and WW are each

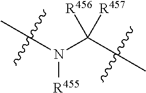

or TT, UU, VV, and WW are each

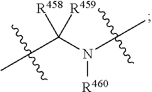

with the proviso when vv is 0 and uu is 1, one of SS and WW is

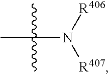

and the other of SS and WW is

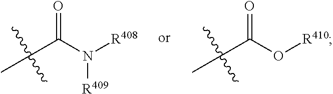

R⁴⁰¹ is

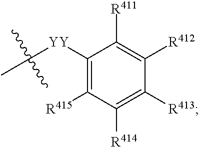

R⁴⁰² is

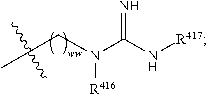

R⁴⁰³ is

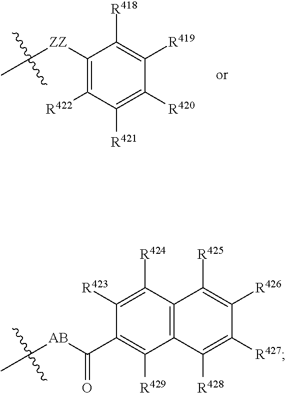

R⁴⁰⁴ is

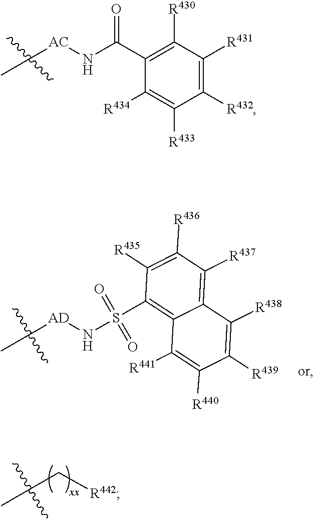

R⁴⁰⁵ is

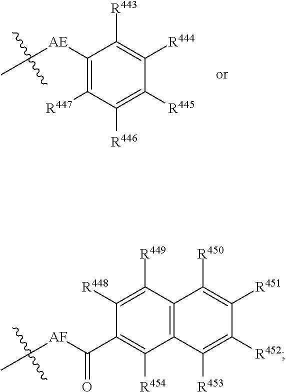

wherein
   R⁴⁰⁶, R⁴⁰⁷, R⁴⁰⁸, R⁴⁰⁹, and R⁴¹⁰ are each independently a hydrogen or substituted or unsubstituted C₁-C₆ alkyl, C₂-C₆ alkenyl, C₂-C₆ alkynyl, saturated or unsaturated cycloalkyl, cycloalkylalkyl, aryl, aralkyl, 5- or 6-membered saturated or unsaturated heterocylyl, heterobicycyl, heteroaryl, or amino protecting group; or R⁴⁰⁶ and R⁴⁰⁷ together form a 3-, 4-, 5-, 6-, 7-, or 8-member substituted or unsubstituted heterocycyl ring;
   R⁴⁵⁵ and R⁴⁶⁰ are at each occurrence independently a hydrogen, —C(O)R^{e}, or an unsubstituted C₁-C₆ alkyl group;
   R⁴⁵⁶ and R⁴⁵⁷ are each independently a hydrogen or substituted or unsubstituted C₁-C₆ alkyl group; or together R⁴⁵⁶ and R⁴⁵⁷ are C═O;
   R⁴⁵⁸ and R⁴⁵⁹ are each independently a hydrogen or substituted or unsubstituted C₁-C₆ alkyl group; or together R⁴⁵⁸ and R⁴⁵⁹ are C═O;
   R⁴¹¹, R⁴¹², R⁴¹³, R⁴¹⁴, R⁴¹⁵, R⁴¹⁸, R⁴¹⁹, R⁴²⁰, R⁴²¹, R⁴²², R⁴²³, R⁴²⁴, R⁴²⁵, R⁴²⁶, R⁴²⁷, R⁴²⁸, R⁴²⁹, R⁴³⁰, R⁴³¹, R⁴³², R⁴³³, R⁴³⁴, R⁴³⁵, R⁴³⁶, R⁴³⁷, R⁴³⁸, R⁴³⁹, R⁴⁴⁰, R⁴⁴¹, R⁴⁴³, R⁴⁴⁴, R⁴⁴⁵, R⁴⁴⁶, R⁴⁴⁷, R⁴⁴⁸, R⁴⁴⁹, R⁴⁵⁰, R⁴⁵¹, R⁴⁵², R⁴⁵³, and R⁴⁵⁴ are each independently a hydrogen, deuterium, amino, amido, —NO₂, —CN, —OR^{e}, —SR^{e}, —NR^{e}R^{e}, —F, —Cl, —Br, —I, or a substituted or unsubstituted C₁-C₆ alkyl, C₁-C₆ alkoxy, —C(O)-alkyl, —C(O)-aryl, —C(O)-aralkyl, —C(O)₂R^{e}, C₁-C₄ alkylamino, C₁-C₄ dialkylamino, or perhaloalkyl group;
   R⁴¹⁶ and R⁴¹⁷ are each independently a hydrogen, —C(O)R^{e}, or a substituted or unsubstituted C₁-C₆ alkyl;
   R⁴⁴² is a hydrogen, —OR^{e}, —SR^{e}, —NR^{e}R^{e}, —NR^{e}R^{f}, —CO₂R^{e}, —C(O)NR^{e}R^{e}, —NR^{e}C(O)R^{e}, —NR^{e}C(NH)NH₂, —NR^{e}-dansyl, or a substituted or unsubstituted alkyl, aryl, or aralkyl group;
   YY, ZZ, and AE are each independently absent, —NH(CO)—, or —CH₂—;
   AB, AC, AD, and AF are each independently absent or C₁-C₆ alkylene group;
   R^{e} at each occurrence is independently a hydrogen or a substituted or unsubstituted C₁-C₆ alkyl group;
   R^{f} at each occurrence is independently a C₁-C₆ alkylene-NR^{e}-dansyl or C₁-C₆ alkylene-NR^{e}-anthraniloyl group;
   rr, ss, and vv are each independently 0 or 1; tt and uu are each 1 with the proviso that rr+ss+tt+uu+vv equals 4 or 5; and
   ww and xx are each independently 1, 2, 3, 4, or 5.

In some embodiments of peptides of Formula III,
R⁴⁰⁶ is a hydrogen, substituted or unsubstituted C₁-C₆ alkyl group,

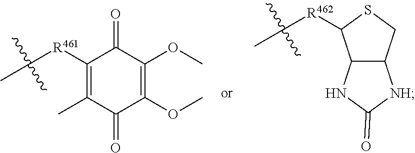

wherein R⁴⁶¹ is a —C₁-C₁₀ alkylene-CO₂— or —CO₂-C₁-C₁₀ alkylene-CO₂—; and R¹⁶² is C₁-C₁₀ alkylene or C₁-C₁₀ alkylene-CO₂—;
R⁴⁰⁷, R⁴⁰⁸, R⁴⁰⁹, and R⁴¹⁰ are each independently a hydrogen or substituted or unsubstituted C₁-C₆ alkyl group;
R⁴⁵⁵ and R⁴⁶⁰ are each independently a hydrogen, —C(O)—C₁-C₆ alkyl, or methyl group;
R⁴⁵⁶ and R⁴⁵⁷ are each a hydrogen or together R⁴⁵⁶ and R⁴⁵⁷ are C═O;
R⁴⁵⁸ and R⁴⁵⁹ are each a hydrogen or together R⁴⁵⁸ and R⁴⁵⁹ are C═O;
R⁴¹⁶ and R⁴¹⁷ are each independently a hydrogen or —C(O)R^{e};
R⁴¹¹, R⁴¹², R⁴¹³, R⁴¹⁴, R⁴¹⁵, R⁴¹⁸, R⁴¹⁹, R⁴²⁰, R⁴²¹, R⁴²², R⁴⁴³, R⁴⁴⁴, R⁴⁴⁵, R⁴⁴⁶, and R⁴⁴⁷, are each independently a hydrogen, deuterium, methyl, or —OR^{e} group;
R⁴²³, R⁴²⁴, R⁴²⁵, R⁴²⁶, R⁴²⁷, R⁴²⁸, R⁴²⁹, R⁴³⁰, R⁴³¹, R⁴³², R⁴³³, R⁴³⁴, R⁴³⁵, R⁴³⁶, R⁴³⁷, R⁴³⁸, R⁴³⁹, R⁴⁴⁰, R⁴⁴¹, R⁴⁴⁸, R⁴⁴⁹, R⁴⁵⁰, R⁴⁵¹, R⁴⁵², R⁴⁵³, and R⁴⁵⁴ are each independently a hydrogen, NR^{e}R^{e}, or substituted or unsubstituted C₁-C₆ alkyl group;
R⁴⁴² is a —NR^{e}R^{e};
YY, ZZ, and AE are each independently absent or —CH₂—;
AB, AC, AD, and AF are each independently absent or C₁-C₄ alkylene group;
R^{e} at each occurrence is independently a hydrogen or a substituted or unsubstituted C₁-C₆ alkyl group;
rr, ss, and vv are each independently 0 or 1; tt and uu are each 1 with the proviso that rr+ss+tt+uu+vv equals 4 or 5; and
ww and xx are each independently 1, 2, 3, 4, or 5.

In some embodiments of peptides of Formula III,

### R⁴⁰⁶ is

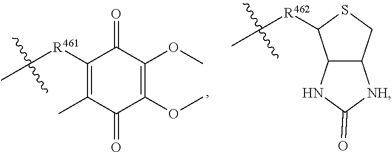

hydrogen, or methyl, wherein R⁴⁶¹ is a —(CH₂)₃—CO₂—, —(CH₂)₉—CO₂—, or —CO₂—(CH₂)₂—CO2— and R⁴⁶² is —(CH₂)₄—CO₂—;
R⁴⁰⁷, R⁴⁰⁸, R⁴⁰⁹, and R⁴¹⁰ are each a hydrogen or methyl group;
R⁴⁵⁵ and R⁴⁶⁰ are each independently a hydrogen, —C(O)CH₃, or methyl group;
R⁴⁵⁶ and R⁴⁵⁷ are each a hydrogen or together R⁴⁵⁶ and R⁴⁵⁷ are C═O;
R⁴⁵⁸ and R⁴⁵⁹ are each a hydrogen or together R⁴⁵⁸ and R⁴⁵⁹ are C═O;
R⁴¹⁶ and R⁴¹⁷ are each independently a hydrogen or —C(O)CH₃;
R⁴²⁶, R⁴³⁸, and R⁴⁵¹ are each —N(CH₃)₂;
R⁴³⁴ and R⁴⁴² are each —NH₂;
R⁴²³, R⁴²⁴, R⁴²⁵, R⁴²⁷, R⁴²⁸, R⁴²⁹, R⁴³⁰, R⁴³¹, R⁴³², R⁴³³, R⁴³⁵, R⁴³⁶, R⁴³⁷, R⁴³⁹, R⁴⁴⁰, R⁴⁴¹, R⁴⁴³, R⁴⁴⁴, R⁴⁴⁵, R⁴⁴⁶, R⁴⁴⁷, R⁴⁴⁸, R⁴⁴⁹, R⁴⁵⁰, R^{452 R}⁴⁵³, and R⁴⁵⁴ are each hydrogen;
R⁴¹², R⁴¹⁴, R⁴¹⁹, and R⁴²¹ are each independently hydrogen or deuterium;
R⁴¹¹, R⁴¹⁵, R⁴¹⁸, and R⁴²² are each independently hydrogen, deuterium, or methyl;
R⁴¹³ and R⁴²⁰ are each independently hydrogen, deuterium, or OR^{e};
YY, ZZ, and AE are each independently —CH₂—;
AB, AC, AD, and AF are each —CH₂— or a butylene group;
R^{e} at each occurrence is independently a hydrogen or a substituted or unsubstituted C₁-C₆ alkyl group;
rr, ss, and vv are each independently 0 or 1; tt and uu are each 1 with the proviso that rr+ss+tt+uu+vv equals 4 or 5; and
ww and xx are each independently 3 or 4.

In certain embodiments of Formula III,

SS is

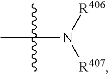

XX is

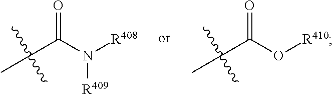

TT, UU, VV, and WW are each independently

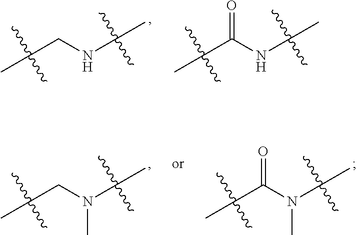

with the proviso when vv is 0 and uu is 1, WW is

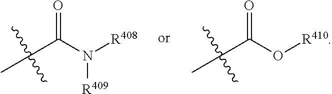

In some embodiments, the peptide of Formula III is selected from the peptides shown in Table D.

TABLE D
6-Butyric acid CoQ0-Phe-D-Arg-Phe-Lys-NH₂
6-Decanoic acid CoQ0-Phe-D-Arg-Phe-Lys-NH₂
H-D-N2-acetylarginine-Dmt-Lys-Phe-NH₂
H-D-N8-acetylarginine-Dmt-Lys-Phe-NH₂
H-N2-acetyl-D-arginine-L-Dmt-L-Lys-L-Phe-NH₂
H-N7-acetyl-D-arginine-Dmt-Lys-Phe-NH₂
H-Phe(d5)-D-Arg-Phe(d5)-Lys-NH₂
Succinic monoester CoQ0-Phe-D-Arg-Phe-Lys-HN₂
Dmt-D-Arg-Phe-(atn)Dap-NH₂
Dmt-D-Arg-Phe-(dns)Dap-NH₂
Dmt-D-Arg-Ald-Lys-NH₂
Dmt-D-Arg-Phe-Lys-Ald-NH₂
Bio-2′6′Dmt-D-Arg-Phe-Lys-NH₂
2′6′Dmt-D-Arg-Phe-dnsDap-NH₂
2′6′Dmt-D-Arg-Phe-atnDap-NH₂
H-D-Arg-Ψ[CH₂-NH]Dmt-Lys-Phe-NH₂
H-D-Arg-Dmt-Ψ[CH₂-NH]Lys-Phe-NH₂
H-D-Arg-Dmt-LysΨ[CH₂-NH]Phe-NH₂
H-D-Arg-Dmt-Ψ[CH₂-NH]Lys-Ψ[CH₂-NH]Phe-NH₂

In some embodiments, the peptide is selected from the peptides shown in Table E.

TABLE E
Arg-D-Leu-D-Tyr-Phe-Lys-Glu-D-Lys-Arg-D-Trp-Lys-
D-Phe-Tyr-D-Arg-Gly
Asp-Arg-D-Phe-Cys-Phe-D-Arg-D-Lys-Tyr-Arg-D-Tyr-
Trp-D-His-Tyr-D-Phe-Lys-Phe
D-Glu-Asp-Lys-D-Arg-D-His-Phe-Phe-D-Val-Tyr-Arg-
Tyr-D-Tyr-Arg-His-Phe-NH₂
Glu-Arg-D-Lys-Tyr-D-Val-Phe-D-His-Trp-Arg-D-Gly-
Tyr-Arg-D-Met-NH₂
Gly-Ala-Lys-Phe-D-Lys-Glu-Arg-Tyr-His-D-Arg-D-Arg-
Asp-Tyr-Trp-D-His-Trp-His-D-Lys-Asp
His-Tyr-D-Arg-Trp-Lys-Phe-D-Asp-Ala-Arg-Cys-D-Tyr-
His-Phe-D-Lys-Tyr-His-Ser-NH₂
Phe-Phe-D-Tyr-Arg-Glu-Asp-D-Lys-Arg-D-Arg-His-Phe-
NH₂
Phe-Tyr-Lys-D-Arg-Trp-His-D-Lys-D-Lys-Glu-Arg-D-
Tyr-Thr
Thr-Tyr-Arg-D-Lys-Trp-Tyr-Glu-Asp-D-Lys-D-Arg-His-
Phe-D-Tyr-Gly-Val-Ile-D-His-Arg-Tyr-Lys-NH₂
Tyr-Asp-D-Lys-Tyr-Phe-D-Lys-D-Arg-Phe-Pro-D-Tyr-
His-Lys
Tyr-D-His-Phe-D-Arg-Asp-Lys-D-Arg-His-Trp-D-His-
Phe
Phe-Tyr-Lys-D-Arg-Trp-His-D-Lys-D-Lys-Glu-Arg-D-
Tyr-Thr
Tyr-Asp-D-Lys-Tyr-Phe-D-Lys-D-Arg-Phe-Pro-D-Tyr-
His-Lys
Glu-Arg-D-Lys-Tyr-D-Val-Phe-D-His-Trp-Arg-D-Gly-
Tyr-Arg-D-Met-NH₂
Arg-D-Leu-D-Tyr-Phe-Lys-Glu-D-Lys-Arg-D-Trp-Lys-D-
Phe-Tyr-D-Arg-Gly
Gly-Ala-Lys-Phe-D-Lys-Glu-Arg-Tyr-His-D-Arg-D-Arg-
Asp-Tyr-Trp-D-His-Trp-His-D-Lys-Asp
Gly-Ala-Lys-Phe-D-Lys-Glu-Arg-Tyr-His-D-Arg-D-Arg-
Asp-Tyr-Trp-D-His-Trp-His-D-Lys-Asp

In one embodiment, the aromatic-cationic peptides of the present technology have a core structural motif of alternating aromatic and cationic amino acids. For example, the peptide may be a tetrapeptide defined by any of Formulas A to F set forth below:

Aromatic—Cationic—Aromatic—Cationic (Formula A)

Cationic—Aromatic—Cationic—Aromatic (Formula B)

Aromatic—Aromatic—Cationic—Cationic (Formula C)

Cationic—Cationic—Aromatic—Aromatic (Formula D)

Aromatic—Cationic—Cationic—Aromatic (Formula E)

Cationic—Aromatic—Aromatic—Cationic (Formula F)

wherein, Aromatic is a residue selected from the group consisting of: Phe (F), Tyr (Y), and Trp (W). In some embodiments, the Aromatic residue may be substituted with a saturated analog of an aromatic residue, e.g., Cyclohexylalanine (Cha). In some embodiments, Cationic is a residue selected from the group consisting of: Arg (R), Lys (K), and His (H).

The amino acids of the aromatic-cationic peptides of the present technology can be any amino acid. As used herein, the term “amino acid” is used to refer to any organic molecule that contains at least one amino group and at least one carboxyl group. In some embodiments, at least one amino group is at the α position relative to the carboxyl group.

The amino acids may be naturally occurring. Naturally occurring amino acids include, for example, the twenty most common levorotatory (L,) amino acids normally found in mammalian proteins, i.e., alanine (Ala), arginine (Arg), asparagine (Asn), aspartic acid (Asp), cysteine (Cys), glutamine (Gln), glutamic acid (Glu), glycine (Gly), histidine (His), isoleucine (Ile), leucine (Leu), lysine (Lys), methionine (Met), phenylalanine (Phe), proline (Pro), serine (Ser), threonine (Thr), tryptophan, (Trp), tyrosine (Tyr), and valine (Val).

Other naturally occurring amino acids include, for example, amino acids that are synthesized in metabolic processes not associated with protein synthesis. For example, the amino acids ornithine and citrulline are synthesized in mammalian metabolism during the production of urea.

The peptides useful in the present technology can contain one or more non-naturally occurring amino acids. The non-naturally occurring amino acids may be (L-), dextrorotatory (D-), or mixtures thereof. In some embodiments, the peptide has no amino acids that are naturally occurring.

Non-naturally occurring amino acids are those amino acids that typically are not synthesized in normal metabolic processes in living organisms, and do not naturally occur in proteins. In certain embodiments, the non-naturally occurring amino acids useful in the present technology are also not recognized by common proteases.

The non-naturally occurring amino acid can be present at any position in the peptide. For example, the non-naturally occurring amino acid can be at the N terminus, the C-terminus, or at any position between the N-terminus and the C-terminus.

The non-natural amino acids may, for example, comprise alkyl, aryl, or alkylaryl groups. Some examples of alkyl amino acids include a-aminobutyric acid, β-aminobutyric acid, γ-aminobutyric acid, δ-aminovaleric acid, and ϵ-aminocaproic acid. Some examples of aryl amino acids include ortho-, meta, and para-aminobenzoic acid. Some examples of alkylaryl amino acids include ortho-, meta-, and para-aminophenyl acetic acid, and γ-phenyl-β-aminobutyric acid.

Non-naturally occurring amino acids also include derivatives of naturally occurring amino acids. The derivatives of naturally occurring amino acids may, for example, include the addition of one or more chemical groups to the naturally occurring amino acid.

For example, one or more chemical groups can be added to one or more of the 2′, 3′, 4′, 5′, or 6′ position of the aromatic ring of a phenylalanine or tyrosine residue, or the 4′, 5′, 6′, or 7′ position of the benzo ring of a tryptophan residue. The group can be any chemical group that can be added to an aromatic ring. Some examples of such groups include branched or unbranched C₁-C₄ alkyl, such as methyl, ethyl, n-propyl, isopropyl, butyl, isobutyl, or t-butyl, C₁-C₄ alkyloxy (i.e., alkoxy), amino, C₁-C₄ alkylamino and C₁-C₄ dialkylamino (e.g., methylamino, dimethylamino), nitro, hydroxyl, halo (i.e., fluoro, chloro, bromo, or iodo). Some specific examples of non-naturally occurring derivatives of naturally occurring amino acids include norvaline (Nva), norleucine (Nle), and hydroxyproline (Hyp).

Another example of a modification of an amino acid in a peptide useful in the present methods is the derivatization of a carboxyl group of an aspartic acid or a glutamic acid residue of the peptide. One example of derivatization is amidation with ammonia or with a primary or secondary amine, e.g., methylamine, ethylamine, dimethylamine or diethylamine. Another example of derivatization includes esterification with, for example, methyl or ethyl alcohol.

Another such modification includes derivatization of an amino group of a lysine, arginine, or histidine residue. For example, such amino groups can be alkylated or acylated. Some suitable acyl groups include, for example, a benzoyl group or an alkanoyl group comprising any of the C₁-C₄ alkyl groups mentioned above, such as an acetyl or propionyl group.

In some embodiments, the non-naturally occurring amino acids are resistant, and in some embodiments insensitive, to common proteases. Examples of non-naturally occurring amino acids that are resistant or insensitive to proteases include the dextrorotatory (D-) form of any of the above-mentioned naturally occurring L-amino acids, as well as L- and/or D non-naturally occurring amino acids. The D-amino acids do not normally occur in proteins, although they are found in certain peptide antibiotics that are synthesized by means other than the normal ribosomal protein synthetic machinery of the cell, as used herein, the D-amino acids are considered to be non-naturally occurring amino acids.

In order to minimize protease sensitivity, the peptides useful in the methods of the present technology should have less than five, less than four, less than three, or less than two contiguous L-amino acids recognized by common proteases, irrespective of whether the amino acids are naturally or non-naturally occurring. In some embodiments, the peptide has only D-amino acids, and no L-amino acids.

If the peptide contains protease sensitive sequences of amino acids, at least one of the amino acids is a non-naturally-occurring D-amino acid, thereby conferring protease resistance. An example of a protease sensitive sequence includes two or more contiguous basic amino acids that are readily cleaved by common proteases, such as endopeptidases and trypsin. Examples of basic amino acids include arginine, lysine and histidine. In some embodiments, at least one of the amides in the peptide backbone is alkylated, thereby conferring protease resistance.

It is important that the aromatic-cationic peptides have a minimum number of net positive charges at physiological pH in comparison to the total number of amino acid residues in the peptide. The minimum number of net positive charges at physiological pH is referred to below as (pₘ). The total number of amino acid residues in the peptide is referred to below as (r).

The minimum number of net positive charges discussed below are all at physiological pH. The term “physiological pH” as used herein refers to the normal pH in the cells of the tissues and organs of the mammalian body. For instance, the physiological pH of a human is normally approximately 7.4, but normal physiological pH in mammals may be any pH from about 7.0 to about 7.8.

Typically, a peptide has a positively charged N-terminal amino group and a negatively charged C-terminal carboxyl group. The charges cancel each other out at physiological pH. As an example of calculating net charge, the peptide Tyr-Arg-Phe-Lys-Glu-His-Trp-Arg has one negatively charged amino acid (i.e., Glu) and four positively charged amino acids (i.e., two Arg residues, one Lys, and one His). Therefore, the above peptide has a net positive charge of three.

In one embodiment, the aromatic-cationic peptides have a relationship between the minimum number of net positive charges at physiological pH (pₘ) and the total number of amino acid residues (r) wherein 3pₘ is the largest number that is less than or equal to r+1. In this embodiment, the relationship between the minimum number of net positive charges (pₘ) and the total number of amino acid residues (r) is as follows:

TABLE 1
Amino acid number and net positive charges (3pₘ ≤ p + 1)

| | (r) | | | | | | | | | | | | | | | | | |
|---|---|---|---|---|---|---|---|---|---|---|---|---|---|---|---|---|---|---|
| | 3 | 4 | 5 | 6 | 7 | 8 | 9 | 10 | 11 | 12 | 13 | 14 | 15 | 16 | 17 | 18 | 19 | 20 |
| (pₘ) | 1 | 1 | 2 | 2 | 2 | 3 | 3 | 3 | 4 | 4 | 4 | 5 | 5 | 5 | 6 | 6 | 6 | 7 |

In another embodiment, the aromatic-cationic peptides have a relationship between the minimum number of net positive charges (pₘ) and the total number of amino acid residues (r) wherein 2pₘ is the largest number that is less than or equal to r+1. In this embodiment, the relationship between the minimum number of net positive charges (pₘ) and the total number of amino acid residues (r) is as follows:

TABLE 2
Amino acid number and net positive charges (2pₘ ≤ p + 1)

| | (r) | | | | | | | | | | | | | | | | | |
|---|---|---|---|---|---|---|---|---|---|---|---|---|---|---|---|---|---|---|
| | 3 | 4 | 5 | 6 | 7 | 8 | 9 | 10 | 11 | 12 | 13 | 14 | 15 | 16 | 17 | 18 | 19 | 20 |
| (pₘ) | 2 | 2 | 3 | 3 | 4 | 4 | 5 | 5 | 6 | 6 | 7 | 7 | 8 | 8 | 9 | 9 | 10 | 10 |

In one embodiment, the minimum number of net positive charges (pₘ) and the total number of amino acid residues (r) are equal. In another embodiment, the peptides have three or four amino acid residues and a minimum of one net positive charge, or a minimum of two net positive charges, or a minimum of three net positive charges.

It is also important that the aromatic-cationic peptides have a minimum number of aromatic groups in comparison to the total number of net positive charges (pₜ). The minimum number of aromatic groups will be referred to below as (a). Naturally-occurring amino acids that have an aromatic group include the amino acids histidine, tryptophan, tyrosine, and phenylalanine. For example, the hexapeptide Lys-Gln-Tyr-D-Arg-Phe-Trp has a net positive charge of two (contributed by the lysine and arginine residues) and three aromatic groups (contributed by tyrosine, phenylalanine and tryptophan residues).

The aromatic-cationic peptides should also have a relationship between the minimum number of aromatic groups (a) and the total number of net positive charges at physiological pH (pₜ) wherein 3a is the largest number that is less than or equal to pₜ+1, except that when pₜ is 1, a may also be 1. In this embodiment, the relationship between the minimum number of aromatic groups (a) and the total number of net positive charges (pₜ) is as follows:

TABLE 3
Aromatic groups and net positive charges (3a ≤ pₜ + 1 or a = pₜ = 1)

| | (pₜ) | | | | | | | | | | | | | | | | | | | |
|---|---|---|---|---|---|---|---|---|---|---|---|---|---|---|---|---|---|---|---|---|
| | 1 | 2 | 3 | 4 | 5 | 6 | 7 | 8 | 9 | 10 | 11 | 12 | 13 | 14 | 15 | 16 | 17 | 18 | 19 | 20 |
| (a) | 1 | 1 | 1 | 1 | 2 | 2 | 2 | 3 | 3 | 3 | 4 | 4 | 4 | 5 | 5 | 5 | 6 | 6 | 6 | 7 |

In another embodiment, the aromatic-cationic peptides have a relationship between the minimum number of aromatic groups (a) and the total number of net positive charges (pₜ) wherein 2a is the largest number that is less than or equal to pₜ+1. In this embodiment, the relationship between the minimum number of aromatic amino acid residues (a) and the total number of net positive charges (pₜ) is as follows:

TABLE 4
Aromatic groups and net positive charges (2a ≤ pₜ + 1 or a = pₜ = 1)

| | (pₜ) | | | | | | | | | | | | | | | | | | | |
|---|---|---|---|---|---|---|---|---|---|---|---|---|---|---|---|---|---|---|---|---|
| | 1 | 2 | 3 | 4 | 5 | 6 | 7 | 8 | 9 | 10 | 11 | 12 | 13 | 14 | 15 | 16 | 17 | 18 | 19 | 20 |
| (a) | 1 | 1 | 2 | 2 | 3 | 3 | 4 | 4 | 5 | 5 | 6 | 6 | 7 | 7 | 8 | 8 | 9 | 9 | 10 | 10 |

In another embodiment, the number of aromatic groups (a) and the total number of net positive charges (pt) are equal.

In some embodiments, carboxyl groups, especially the terminal carboxyl group of a C-terminal amino acid, are amidated with, for example, ammonia to form the C-terminal amide. Alternatively, the terminal carboxyl group of the C-terminal amino acid may be amidated with any primary or secondary amine. The primary or secondary amine may, for example, be an alkyl, especially a branched or unbranched C₁-C₄ alkyl, or an aryl amine. Accordingly, the amino acid at the C-terminus of the peptide may be converted to an amido, N-methylamido, N-ethylamido, N,N-dimethylamido, N,N-diethyl amido, N-methyl-N-ethylamido, N-phenylamido or N-phenyl-N-ethylamido group.

The free carboxylate groups of the asparagine, glutamine, aspartic acid, and glutamic acid residues not occurring at the C-terminus of the aromatic-cationic peptides of the present technology may also be amidated wherever they occur within the peptide. The amidation at these internal positions may be with ammonia or any of the primary or secondary amines described herein.

In one embodiment, the aromatic-cationic peptide useful in the methods of the present technology is a tripeptide having two net positive charges and at least one aromatic amino acid. In a particular embodiment, the aromatic-cationic peptide useful in the methods of the present technology is a tripeptide having two net positive charges and two aromatic amino acids.

In some embodiments, the aromatic-cationic peptide is a peptide having:

at least one net positive charge;

a minimum of four amino acids;

a maximum of about twenty amino acids;

a relationship between the minimum number of net positive charges (pₘ) and the total number of amino acid residues (r) wherein 3pₘ is the largest number that is less than or equal to r+1; and a relationship between the minimum number of aromatic groups (a) and the total number of net positive charges (pₜ) wherein 2a is the largest number that is less than or equal to pₜ+1, except that when a is 1, pₜ may also be 1.

In one embodiment, 2pₘ is the largest number that is less than or equal to r+1, and a may be equal to pₜ. The aromatic-cationic peptide may be a water-soluble peptide having a minimum of two or a minimum of three positive charges.

In one embodiment, the peptide comprises one or more non-naturally occurring amino acids, for example, one or more D-amino acids. In some embodiments, the C-terminal carboxyl group of the amino acid at the C-terminus is amidated. In certain embodiments, the peptide has a minimum of four amino acids. The peptide may have a total of about 6, a total of about 9, or a total of about 12 amino acids.

In one embodiment, the peptides have a tyrosine residue or a tyrosine derivative at the N-terminus (i.e., the first amino acid position). Suitable derivatives of tyrosine include 2′-methyltyrosine (Mmt); 2′,6′-dimethyltyrosine (2′6′-Dmt); 3′,5′-dimethyltyrosine (3′5′Dmt); N,2′,6′-trimethyltyrosine (Tmt); and 2′-hydroxy-6′-methyltyrosine (Hmt).

In one embodiment, a peptide has the formula Tyr-D-Arg-Phe-Lys-NH₂. Tyr-D-Arg-Phe-Lys-NH₂ has a net positive charge of three, contributed by the amino acids tyrosine, arginine, and lysine and has two aromatic groups contributed by the amino acids phenylalanine and tyrosine. The tyrosine of Tyr-D-Arg-Phe-Lys-NH₂ can be a modified derivative of tyrosine such as in 2′,6′-dimethyltyrosine to produce the compound having the formula 2′,6′-Dmt-D-Arg-Phe-Lys-NH₂. 2′,6′-Dmt-D-Arg-Phe-Lys-NH₂ has a molecular weight of 640 and carries a net three positive charge at physiological pH. 2′,6′-Dmt-D-Arg-Phe-Lys-NH₂ readily penetrates the plasma membrane of several mammalian cell types in an energy-independent manner (Zhao et al., J. Pharmacol Exp Ther., 304:425-432, 2003).

Alternatively, in some embodiments, the aromatic-cationic peptide does not have a tyrosine residue or a derivative of tyrosine at the N-terminus (i.e., amino acid position 1). The amino acid at the N-terminus can be any naturally-occurring or non-naturally-occurring amino acid other than tyrosine. In one embodiment, the amino acid at the N-terminus is phenylalanine or its derivative. Exemplary derivatives of phenylalanine include 2′-methylphenylalanine (Mmp), 2′,6′-dimethylphenylalanine (2′,6′-Dmp), N,2′,6′-trimethylphenylalanine (Tmp), and 2′-hydroxy-6′-methylphenylalanine (Hmp).

An example of an aromatic-cationic peptide that does not have a tyrosine residue or a derivative of tyrosine at the N-terminus is a peptide with the formula Phe-D-Arg-Phe-Lys-NH₂. Alternatively, the N-terminal phenylalanine can be a derivative of phenylalanine such as 2′,6′-dimethylphenylalanine (2′6′-Dmp). In one embodiment, the amino acid sequence of 2′,6′-Dmt-D-Arg-Phe-Lys-NH₂ is rearranged such that Dmt is not at the N-terminus. An example of such an aromatic-cationic peptide is a peptide having the formula of D-Arg-2′6′-Dmt-Lys-Phe-NH₂.

Suitable substitution variants of the peptides listed herein include conservative amino acid substitutions. Amino acids may be grouped according to their physicochemical characteristics as follows:

(a) Non-polar amino acids: Ala(A) Ser(S) Thr(T) Pro(P) Gly(G) Cys (C);

(b) Acidic amino acids: Asn(N) Asp(D) Glu(E) Gln(Q);

(c) Basic amino acids: His(H) Arg(R) Lys(K);

(d) Hydrophobic amino acids: Met(M) Leu(L) Ile(I) Val(V); and

(e) Aromatic amino acids: Phe(F) Tyr(Y) Trp(W).

Substitutions of an amino acid in a peptide by another amino acid in the same group are referred to as a conservative substitution and may preserve the physicochemical characteristics of the original peptide. In contrast, substitutions of an amino acid in a peptide by another amino acid in a different group are generally more likely to alter the characteristics of the original peptide.

The amino acids of the peptides disclosed herein may be in either the L- or the D-configuration.

The peptides may be synthesized by any of the methods well known in the art. Suitable methods for chemically synthesizing the protein include, for example, those described by Stuart and Young in Solid Phase Peptide Synthesis, Second Edition, Pierce Chemical Company (1984), and in Methods Enzymol., 289, Academic Press, Inc., New York (1997).

### Pulmonary Arterial Hypertension (PAH)

Pulmonary arterial hypertension (PAH) is a chronic and progressive disease of the lung vascular system in which endothelial dysfunction and vascular remodeling of endothelial and smooth muscle cells lead to the obstruction of pulmonary arteries, resulting in increased pulmonary vascular resistance and pulmonary arterial pressures.

Pulmonary hypertension in humans is defined by right-heart catheterization (RHC) showing a sustained elevation of mean pulmonary arterial pressure (mPAP) greater than or equal to 25 mm Hg at rest or greater than or equal to 30 mm Hg with exercise, with normal pulmonary artery occlusion pressure (i.e., pulmonary-capillary wedge pressure or left ventricular end-diastolic pressure) of less than or equal to 15 mm Hg.

PAH may occur in isolation or in association with several clinical conditions. According to the clinical classification of pulmonary hypertension from the Fifth World Symposium, PAH can occur in an idiopathic form (IPAH), heritable/familial form (FPAH), or associated with other medical conditions (APAH), such as connective tissue disease, portal hypertension, HIV infection, schistosomiasis, and pulmonary capillary hemangiomatosis. PAH has also been associated with drug and toxin exposure. PAH may also be caused by pulmonary veno-occlusive disease and is related to persistent pulmonary hypertension of the newborn (PPHN).

Idiopathic and Heritable PAH. Idiopathic PAH refers to a sporadic disease with neither a family history of PAH nor an identified risk factor. Heritable or familial forms of PAH (FPAH) are most often associated with germline mutations in the bone morphogenetic protein receptor 2 (BMPR²) gene. (See Lai, et al. Circ. Res. 115:115-130 (2014)). Up to 80% of familial cases of PAH have been linked to germline mutations in BMPR², which encodes for a protein that is a member of the transforming growth factor TGF-β signaling family. Bone morphogenetic proteins (BMPs) are multifunctional regulators that modulate cell proliferation, differentiation, and apoptosis in different tissues. Loss of BMPR² in pulmonary arterial endothelial cells increases susceptibility of endothelial cells to apoptosis, which leads to endothelial dysfunction and subsequent development of PAH. More rarely, mutations in activin receptor like kinase type 1 (ALKI) or endoglin (ENG) genes, which encode for components of the BMP signaling pathway, have been identified in patients with PAH. Mutations in the SMAD9, CAV1, and KCNK3 genes are also associated with PAH.

Associated PAH. The most common type of PAH, is caused by or occurs at the same time as other medical conditions such as connective tissue disease, HIV infection, portal hypertension, schistosomiasis, pulmonary veno-occlusive disease, pulmonary capillary hemangiomatosis, persistent pulmonary hypertension of the newborn, and drug- or toxin-induced PAH.

Connective tissue disease (CTD)-associated PAH accounts for 15% to 25% of all PAH cases, with systemic sclerosis and systemic lupus erythematosus as leading causes (Humbert, et al. Am. J. Respir. Crit. Care Med. 173:1023-1030 (2006); Badesch, et al. Chest 137:376-387 (2010)).

Patients with human immunodeficiency virus (HIV) are at an increased risk of developing PAH. The prevalence of PAH in HIV patients is estimated to be 0.5% (Degano, et al. Semin. Respir. Crit. Care Med. 30:440-447 (2009); Sitbon, et al. Am. J. Respir. Crit. Care Med. 177:108-113 (2008)).

Portal hypertension patients develop PAH at a prevalence of about 6%. Portopulmonary hypertension represents a significant problem for liver transplantation because its presence corresponds to increased mortality during and after the procedure.

PAH represents one of the most severe complications of chronic schistosomiasis, an infectious disease caused by parasitic trematode worms. In endemic countries, it is estimated that up to 30% of all PAH patients have schistosomiasis-associated PAH (dos Santos Fernandes, et al. J. Am. Coll. Cardiol. 56:715-720 (2009)).

Pulmonary veno-occlusive disease, pulmonary capillary hemangiomatosis, and persistent pulmonary hypertension of the newborn (PPHN) are also associated with the development of PAH.

Drug- and toxin-induced PAH. A variety of substances have been described as potentially associated with the development of PAH. Exposure to aminorex and fenfluramine derivatives, benfluorex, and toxic rapeseed oil are known risk factors for the development of PAH. Dasatinib, L-tryptophan, methamphetamine, cocaine, and type 1 interferons have also been linked to an increased risk of developing PAH.

### Pathogenesis

In all forms of PAH, the progressive vasculopathy is characterized by an imbalance of vasodilators, such as nitric oxide (NO) and prostacyclin, and vasoconstrictors, such as endothelin-1 (ET-1) and thromboxane A₂. This condition likely precedes the development of aberrant cellular proliferation.

Patients with PAH have impaired NO production associated with diminished eNOS expression, promoting pulmonary vasoconstriction and excessive medial proliferation.

Prostacyclin is a potent vasodilator that binds to its specific I-prostanoid receptor in smooth muscle cells to promote relaxation and subsequent vasodilation. Prostacyclin also attenuates vascular smooth muscle cell proliferation and inhibits platelet aggregation.

Endothelin (ET-1) is a potent vasoconstrictor. ET-1 acts at two different G-protein-coupled receptors: ET_{A} and ET_{B}. As PAH progresses, the cellular distribution of the ET-1 receptors changes, with increased expression of both constrictive ET_{A} and ET_{B} on smooth muscle cells and decreased expression of vasodilatory endothelial ET_{B}. Patients and animals with PAH exhibit increased ET-1 levels in lungs and in circulation, and plasma levels of ET-1 are elevated in patients with PAH.

The histologic appearance of lung tissue in PAH comprises intimal fibrosis, increased medial thickness, intimal hyperplasia of muscular arteries, thrombotic lesions, pulmonary arteriolar occlusion, and plexiform lesions, which lead to a widespread narrowing and obliteration of the pulmonary arteriolar bed.

As PAH progresses, the right ventricle (RV) initially adapts to the increased afterload by a compensatory concentric RV hypertrophy (RVH). The right ventricle is normally part of a low pressure system, with pressures approximately one-sixth of those that the left ventricle encounters. Although RVH initially provides a beneficial compensatory response, eventually, the response becomes more maladaptive and the RV becomes thinned, fibrosed, and dilated, failing to maintain cardiac output. As the right heart fails, blood flowing through the lungs and to the left ventricle decreases. As a result, the left side of the heart may not be able to supply sufficient amounts of oxygenated blood to the body, particularly during physical activity.

### Clinical Manifestations

The clinical signs and symptoms of PAH including persistent dyspnea on exertion, chest pain, light-headedness, exertional presyncope/syncope, palpitations, fatigue, weakness, hoarseness in the voice due to compression of the left laryngeal nerve by the dilated pulmonary artery, venous jugular distension, hepato jugular reflux, hepatomegaly, hepatalgia, lower limb edema, ascites, and generalized edema are almost entirely related to a progressive decline in right heart function.

Analysis of lung tissue in PAH reveals intimal fibrosis, increased medial thickness, intimal hyperplasia of muscular arteries, thrombotic lesions, pulmonary arteriolar occlusion, pulmonary vascular pruning, and plexiform lesions, which lead to a widespread narrowing or obliteration of the pulmonary arteriolar bed.

Elevated serum or plasma levels of brain natriuretic peptide (BNP) (>180 pg/mL) and N-terminal fragment of proBNP (NT-proBNP) (≥1400 pg/mL) serve as biomarkers of dysfunction of the right ventricle in PAH.

### Diagnosis

The diagnostic process of PAH requires a series of investigations. The detection of pulmonary hypertension requires methods including history, physical examination, electrocardiography (ECG), chest radiograph, and trans-thoracic echocardiogram. Exercise testing and hemodynamics are required for evaluation of PAH severity, and right heart catheterization (RHC) confirms the definite diagnosis.

A chest radiograph may indicate right heart enlargement and abnormal lung vessels. In the majority of idiopathic PAH patients, chest radiography is abnormal at the time of diagnosis. Chest radiography findings include central pulmonary arterial dilatation, right atrial and ventricular enlargement.

Electrocardiogram (ECG) provides suggestive evidence of pulmonary hypertension by demonstrating right ventricular hypertrophy and strain, and right atrial dilation. ECGs of patients with PAH frequently show alterations in heart rhythm and changes compatible with right ventricular hypertrophy.

Cardiopulmonary exercise testing (CPET) has been shown to be useful in assessing the severity and prognosis of PAH. CPET findings include: failure to perfuse the ventilated lung, leading to an increase of physiologic dead space and ventilator requirement; failure to increase cardiac output appropriately in response to exercise, causing early lactic acidosis; and exercise-induced hypoxemia increasing the hypoxic ventilator drive.

Right heart catheterization (RHC) is required to confirm the diagnosis of PAH. In normal individuals the average systolic and diastolic pressures in the pulmonary artery are about 25 and 10 mm Hg, respectively, and the mean pulmonary arterial pressure (mPAP) is about 15 mm Hg. PAH is diagnosed by a showing of a sustained elevation of mPAP of greater than or equal to 25 mm Hg at rest or greater than or equal to 30 mm Hg with exercise, with normal pulmonary artery occlusion pressure (i.e., pulmonary-capillary wedge pressure or left ventricular end-diastolic pressure) of less than or equal to 15 mm Hg. The vascular resistance is at the pulmonary arterioles and capillaries and this defines patients with PAH.

### Prognostic Indicators

Methods for assessing the signs, symptoms, or complications of PAH are known in the art. Exemplary methods for assaying the signs, symptoms, or complications of PAH include, but are not limited to, the 6-minute walk test (6MWT), right heart catheterization (RHC) to measure mean pulmonary arterial pressure (mPAP) and pulmonary wedge pressure, serum or plasma brain natriuretic peptide (BNP) and/or N-terminal fragment of proBNP (NT-proBNP) measurements performed using standard laboratory techniques, Doppler echocardiography, cardiac magnetic resonance imaging (MRI), and angiography.

The 6MWT measures the distance one can walk in 6 minutes. A normal 6MWT is >600 to 700 m. A distance <300 to 350 m predicts worse outcome in patients with PAH, and a value of <165 m reflects extremely severe limitation. With respect to improvement in walk distance, a distance of >33 m is associated with improvement in quality-of-life measures.

BNP is a 32-amino acid peptide hormone, which is secreted by cardiomyocytes in response to ventricular stretch, and high levels of which reflect right atrial/ventricular volume and pressure overload. BNP is secreted along with a 76-amino acid NT-proBNP that is biologically inactive. BNP and NT-proBNP are clinically recommended biomarkers of dysfunction of the right ventricle in PAH. A normal BNP level in humans is <100 pg/mL. A normal NT-proBNP level in humans is <300 pg/mL. BNP levels >180 pg/mL are independently associated with mortality. Elevated BNP levels predict diminished exercise tolerance and have been shown to directly correlate with 6-minute walk distance, mean pulmonary arterial pressure (mPAP), pulmonary vascular resistance (PVR), and right atrial pressure (RAP). Similar to BNP, serum or plasma NT-proBNP levels directly correlate with hemodynamic parameters, such as PVR and RAP, and values >1400 ng/mL are associated with reduced survival in PAH patients. Decreases in BNP or NT-proBNP during PAH therapy are associated with improved survival.

Doppler echocardiography provides a noninvasive assessment of the structure and function of the right ventricle, and may be used to monitor progression and response to therapy. Doppler echocardiography can be used to measure right ventricular and atrial pressure, and the degree of septal shift toward the left ventricle in diastole.

Cardiac MRI can be used to assess the size and volume of the right ventricle, cardiac output, and pulmonary artery distensibility and function.

Angiography is a tool that assesses the degree of peripheral vascular pruning seen as the hallmark of obliterative remodeling of PAH. This direct visualization of the branching pattern and vasculopathy illustrates the severity of the disease process but can also be used to evaluate response to therapy.

### Therapeutic Methods

The following discussion is presented by way of example only, and is not intended to be limiting.

One aspect of the present technology includes methods of treating PAH in a subject diagnosed as having, suspected as having, or at risk of having PAH. In therapeutic applications, compositions or medicaments comprising an aromatic-cationic peptide, such as 2′6′-Dmt-D-Arg-Phe-Lys-NH₂, Phe-D-Arg-Phe-Lys-NH₂, or D-Arg-2′6′-Dmt-Lys-Phe-NH₂, or a pharmaceutically acceptable salt thereof, such as acetate, tartrate, or trifluoroacetate salt, are administered to a subject suspected of, or already suffering from such a disease (such as, e.g., subjects exhibiting elevated blood levels of BNP and/or NT-proBNP compared to a normal control subject), in an amount sufficient to cure, or at least partially arrest, the symptoms of the disease, including its complications and intermediate pathological phenotypes in development of the disease.

Subjects suffering from PAH can be identified by any or a combination of diagnostic or prognostic assays known in the art. For example, typical symptoms of PAH include, but are not limited to persistent dyspnea on exertion, chest pain, light-headedness, exertional presyncope/syncope, palpitations, fatigue, weakness, hoarseness in the voice due to compression of the left laryngeal nerve by the dilated pulmonary artery, venous jugular distension, hepato jugular reflux, hepatomegaly, hepatalgia, lower limb edema, ascites, generalized edema, intimal fibrosis of pulmonary arteries, increased medial thickness of pulmonary arteries, intimal hyperplasia of muscular pulmonary arteries, pulmonary artery thrombotic lesions, pulmonary arteriolar occlusion, pulmonary vascular pruning, plexiform lesions in pulmonary arteries, elevated serum or plasma brain natriuretic peptide (BNP) (>180 pg/mL), and elevated serum or plasma N-terminal fragment of proBNP (NT-proBNP) (≥1400 pg/mL).

In some embodiments, the subject may exhibit elevated blood levels of BNP and/or NT-proBNP compared to a normal control subject, which is measureable using techniques known in the art.

In some embodiments, PAH subjects treated with the aromatic-cationic peptide will show amelioration or elimination of one or more of the following symptoms persistent dyspnea on exertion, chest pain, light-headedness, exertional presyncope/syncope, palpitations, fatigue, weakness, hoarseness in the voice due to compression of the left laryngeal nerve by the dilated pulmonary artery, venous jugular distension, hepato jugular reflux, hepatomegaly, hepatalgia, lower limb edema, ascites, generalized edema, intimal fibrosis of pulmonary arteries, increased medial thickness of pulmonary arteries, intimal hyperplasia of muscular pulmonary arteries, pulmonary artery thrombotic lesions, pulmonary arteriolar occlusion, pulmonary vascular pruning, plexiform lesions in pulmonary arteries, elevated serum or plasma brain natriuretic peptide (BNP) (>180 pg/mL), and elevated serum or plasma N-terminal fragment of proBNP (NT-proBNP) (≥1400 pg/mL).

In certain embodiments, PAH subjects treated with the aromatic-cationic peptide will show normalization of BNP and/or NT-proBNP blood levels by at least 5%, at least 10%, at least 25%, at least 50%, at least 75%, or at least 90% compared to untreated PAH subjects. In certain embodiments, PAH subjects treated with the aromatic-cationic peptide will show BNP and/or NT-proBNT blood levels that are similar to that observed in a normal control subject.

### Prophylactic Methods

In one aspect, the present technology provides a method for preventing or delaying the onset of PAH or symptoms of PAH in a subject at risk of having PAH.

Subjects at risk for elevated blood levels of BNP and/or NT-proBNP compared to a normal control subject or at risk for PAH can be identified by, e.g., any or a combination of diagnostic or prognostic assays known in the art. In prophylactic applications, pharmaceutical compositions or medicaments of aromatic-cationic peptides, such as 2′6′-Dmt-D-Arg-Phe-Lys-NH₂, Phe-D-Arg-Phe-Lys-NH₂, or D-Arg-2′6′-Dmt-Lys-Phe-NH₂, or a pharmaceutically acceptable salt thereof, such as acetate, tartrate, or trifluoroacetate salt, are administered to a subject susceptible to, or otherwise at risk of a disease or condition such as e.g., PAH, in an amount sufficient to eliminate or reduce the risk, or delay the onset of the disease, including biochemical, histologic and/or behavioral symptoms of the disease, its complications and intermediate pathological phenotypes presenting during development of the disease. Administration of a prophylactic aromatic-cationic peptide can occur prior to the manifestation of symptoms characteristic of the disease or disorder, such that the disease or disorder is prevented or, alternatively, delayed in its progression.

Subjects at risk for elevated blood levels of BNP and/or NT-proBNP compared to a normal control subject or PAH include, but are not limited to, subjects harboring mutations in the BMPR2, ALK1, ENG, SMAD9, CAV1, or KCNK3 genes. Subjects at risk for elevated blood levels of BNP and/or NT-proBNP compared to a normal control subject or PAH also include, but are not limited to, subjects with connective tissue disease, HIV infection, portal hypertension, schistosomiasis, pulmonary veno-occlusive disease, pulmonary capillary hemagiomatosis, and persistent pulmonary hypertension of the newborn (PPHN). Subjects at risk for elevated blood levels of BNP and/or NT-proBNP compared to a normal control subject or PAH also include, but are not limited to, subjects exposed to various drugs and/or toxins including aminorex and fenfluramine derivatives, benfluorex, toxic rapeseed oil dasatinib, L-tryptophan, methamphetamine, cocaine, and type 1 interferons.

For therapeutic and/or prophylactic applications, a composition comprising an aromatic-cationic peptide, such as 2′6′-Dmt-D-Arg-Phe-Lys-NH₂, Phe-D-Arg-Phe-Lys-NH₂, or D-Arg-2′6′-Dmt-Lys-Phe-NH₂, or a pharmaceutically acceptable salt thereof, such as acetate, tartrate, or trifluoroacetate salt, is administered to the subject. In some embodiments, the peptide composition is administered one, two, three, four, or five times per day. In some embodiments, the peptide composition is administered more than five times per day. Additionally or alternatively, in some embodiments, the peptide composition is administered every day, every other day, every third day, every fourth day, every fifth day, or every sixth day. In some embodiments, the peptide composition is administered weekly, bi-weekly, tri-weekly, or monthly. In some embodiments, the peptide composition is administered for a period of one, two, three, four, or five weeks. In some embodiments, the peptide is administered for six weeks or more. In some embodiments, the peptide is administered for twelve weeks or more. In some embodiments, the peptide is administered for a period of less than one year. In some embodiments, the peptide is administered for a period of more than one year.

In some embodiments of the methods of the present technology, the aromatic-cationic peptide is administered daily for 1 week or more. In some embodiments of the methods of the present technology, the aromatic-cationic peptide is administered daily for 2 weeks or more. In some embodiments of the methods of the present technology, the aromatic-cationic peptide is administered daily for 3 weeks or more. In some embodiments of the methods of the present technology, the aromatic-cationic peptide is administered daily for 4 weeks or more. In some embodiments of the methods of the present technology, the aromatic-cationic peptide is administered daily for 6 weeks or more. In some embodiments of the methods of the present technology, the aromatic-cationic peptide is administered daily for 12 weeks or more.

In some embodiments, treatment with the aromatic-cationic peptide will prevent or delay the onset of one or more of the following symptoms: persistent dyspnea on exertion, chest pain, light-headedness, exertional presyncope/syncope, palpitations, fatigue, weakness, hoarseness in the voice due to compression of the left laryngeal nerve by the dilated pulmonary artery, venous jugular distension, hepato jugular reflux, hepatomegaly, hepatalgia, lower limb edema, ascites, generalized edema, intimal fibrosis of pulmonary arteries, increased medial thickness of pulmonary arteries, intimal hyperplasia of muscular pulmonary arteries, pulmonary artery thrombotic lesions, pulmonary arteriolar occlusion, pulmonary vascular pruning, plexiform lesions in pulmonary arteries, elevated serum or plasma brain natriuretic peptide (BNP) (>180 pg/mL), and elevated serum or plasma N-terminal fragment of proBNP (NT-proBNP) (≥1400 pg/mL). In certain embodiments, the blood levels of BNP and/or NT-proBNP in PAH subjects treated with the aromatic-cationic peptide will resemble those observed in healthy controls.

### Determination of the Biological Effect of the Aromatic-Cationic Peptide-Based Therapeutic

In various embodiments, suitable in vitro or in vivo assays are performed to determine the effect of a specific aromatic-cationic peptide-based therapeutic and whether its administration is indicated for treatment. In various embodiments, in vitro assays can be performed with representative animal models, to determine if a given aromatic-cationic peptide-based therapeutic exerts the desired effect on reducing or eliminating signs and/or symptoms of PAH. Compounds for use in therapy can be tested in suitable animal model systems including, but not limited to rats, mice, chicken, sheep, dogs, cows, monkeys, rabbits, and the like, prior to testing in human subjects. Similarly, for in vivo testing, any of the animal model systems known in the art can be used prior to administration to human subjects. In some embodiments, in vitro or in vivo testing is directed to the biological function of 2′6′-Dmt-D-Arg-Phe-Lys-NH₂, Phe-D-Arg-Phe-Lys-NH₂, or D-Arg-2′6′-Dmt-Lys-Phe-NH₂, or a pharmaceutically acceptable salt thereof, such as acetate, tartrate, or trifluoroacetate salt.

Animal models of PAH are known in the art, including, for example monocrotaline-treated (MCT) rats or dogs, L1cre(+);Bmpr2^{f/f} mice, vasoactive intestinal peptide (VIP) knockout (VIP^{-/-}) mice, and ligation of ductus arteriosus in lambs. See Werchan, et al, Am. J. Physiol. 256:H1328-1336 (1989); Hong, et al. Circulation 118:722-730 (2008); Said, et al. Circulation 115:1260-1268 (2007); de la Roque, et al. Pharmacology & Therapeutics 126:186-199 (2010). Such models may be used to demonstrate the biological effect of aromatic-cationic peptides of the present technology, such as 2′6′-Dmt-D-Arg-Phe-Lys-NH₂, Phe-D-Arg-Phe-Lys-NH₂, or D-Arg-2′6′-Dmt-Lys-Phe-NH₂, in the prevention and treatment of conditions arising from disruption of a particular gene, and for determining what comprises a therapeutically effective amount of peptide in a given context.

### Modes of Administration and Effective Dosages

Any method known to those in the art for contacting a cell, organ or tissue with an aromatic-cationic peptide of the present technology, such as 2′6′-Dmt-D-Arg-Phe-Lys-NH₂, Phe-D-Arg-Phe-Lys-NH₂, or D-Arg-2′6′-Dmt-Lys-Phe-NH₂, or a pharmaceutically acceptable salt thereof, such as acetate, tartrate, or trifluoroacetate salt, may be employed. Suitable methods include in vitro, ex vivo, or in vivo methods. In vivo methods typically include the administration of an aromatic-cationic peptide, such as those described above, to a mammal, suitably a human. When used in vivo for therapy, the aromatic-cationic peptides, such as 2′6′-Dmt-D-Arg-Phe-Lys-NH₂, Phe-D-Arg-Phe-Lys-NH₂, or D-Arg-2′6′-Dmt-Lys-Phe-NH₂, or a pharmaceutically acceptable salt thereof, such as acetate, tartrate, or trifluoroacetate salt, are administered to the subject in effective amounts (i.e., amounts that have desired therapeutic effect). The dose and dosage regimen will depend upon the degree of the infection in the subject, the characteristics of the particular aromatic-cationic peptide used, e.g., its therapeutic index, the subject, and the subject's history.

The effective amount may be determined during pre-clinical trials and clinical trials by methods familiar to physicians and clinicians. An effective amount of a peptide useful in the methods may be administered to a mammal in need thereof by any of a number of well-known methods for administering pharmaceutical compounds. The peptide may be administered systemically or locally.

The peptide may be formulated as a pharmaceutically acceptable salt. The term “pharmaceutically acceptable salt” means a salt prepared from a base or an acid which is acceptable for administration to a patient, such as a mammal (e.g., salts having acceptable mammalian safety for a given dosage regime). However, it is understood that the salts are not required to be pharmaceutically acceptable salts, such as salts of intermediate compounds that are not intended for administration to a patient. Pharmaceutically acceptable salts can be derived from pharmaceutically acceptable inorganic or organic bases and from pharmaceutically acceptable inorganic or organic acids. In addition, when a peptide contains both a basic moiety, such as an amine, pyridine or imidazole, and an acidic moiety such as a carboxylic acid or tetrazole, zwitterions may be formed and are included within the term “salt” as used herein. Salts derived from pharmaceutically acceptable inorganic bases include ammonium, calcium, copper, ferric, ferrous, lithium, magnesium, manganic, manganous, potassium, sodium, and zinc salts, and the like. Salts derived from pharmaceutically acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperadine, polyamine resins, procaine, purines, theobromine, triethylamine, trimethylamine, tripropylamine, tromethamine and the like. Salts derived from pharmaceutically acceptable inorganic acids include salts of boric, carbonic, hydrohalic (hydrobromic, hydrochloric, hydrofluoric or hydroiodic), nitric, phosphoric, sulfamic and sulfuric acids. Salts derived from pharmaceutically acceptable organic acids include salts of aliphatic hydroxyl acids (e.g., citric, gluconic, glycolic, lactic, lactobionic, malic, and tartaric acids), aliphatic monocarboxylic acids (e.g., acetic, butyric, formic, propionic and trifluoroacetic acids), amino acids (e.g., aspartic and glutamic acids), aromatic carboxylic acids (e.g., benzoic, p-chlorobenzoic, diphenylacetic, gentisic, hippuric, and triphenylacetic acids), aromatic hydroxyl acids (e.g., o-hydroxybenzoic, p-hydroxybenzoic, 1-hydroxynaphthalene-2-carboxylic and 3-hydroxynaphthalene-2-carboxylic acids), ascorbic, dicarboxylic acids (e.g., fumaric, maleic, oxalic and succinic acids), glucuronic, mandelic, mucic, nicotinic, orotic, pamoic, pantothenic, sulfonic acids (e.g., benzenesulfonic, camphosulfonic, edisylic, ethanesulfonic, isethionic, methanesulfonic, naphthalenesulfonic, naphthalene-1,5-disulfonic, naphthalene-2,6-disulfonic and p-toluenesulfonic acids), xinafoic acid, and the like. In some embodiments, the salt is an acetate, tartrate, or trifluoroacetate salt.

The aromatic-cationic peptides described herein, such as 2′6′-Dmt-D-Arg-Phe-Lys-NH₂, Phe-D-Arg-Phe-Lys-NH₂, or D-Arg-2′6′-Dmt-Lys-Phe-NH₂, or a pharmaceutically acceptable salt thereof, such as acetate, tartrate, or trifluoroacetate salt, can be incorporated into pharmaceutical compositions for administration, singly or in combination, to a subject for the treatment or prevention of a disorder described herein. Such compositions typically include the active agent and a pharmaceutically acceptable carrier. As used herein the term “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions are typically formulated to be compatible with its intended route of administration. Examples of routes of administration include parenteral (e.g., intravenous, intradermal, intraperitoneal or subcutaneous), oral, inhalation, transdermal (topical), intraocular, iontophoretic, and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. For convenience of the patient or treating physician, the dosing formulation can be provided in a kit containing all necessary equipment (e.g., vials of drug, vials of diluent, syringes and needles) for a treatment course (e.g., 7 days of treatment).

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, a composition for parenteral administration must be sterile and should be fluid to the extent that easy syringability exists. It should be stable under the conditions of manufacture and storage and must be preserved against the contaminating action of microorganisms such as bacteria and fungi.

The aromatic-cationic peptide compositions can include a carrier, which can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thiomerasol, and the like. Glutathione and other antioxidants can be included to prevent oxidation. In many cases, it will be advantageous to include isotonic agents, for example, sugars, polyalcohols such as mannitol, sorbitol, or sodium chloride in the composition. Prolonged absorption of the injectable compositions can be brought about by including in the composition an agent that delays absorption, for example, aluminum monostearate or gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressurized container or dispenser, which contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays. For transdermal administration, the active compounds are formulated into ointments, salves, gels, or creams as generally known in the art. In one embodiment, transdermal administration may be performed by iontophoresis.

A therapeutic protein or peptide can be formulated in a carrier system. The carrier can be a colloidal system. The colloidal system can be a liposome, a phospholipid bilayer vehicle. In one embodiment, the therapeutic peptide is encapsulated in a liposome while maintaining peptide integrity. One skilled in the art would appreciate that there are a variety of methods to prepare liposomes. (See Lichtenberg, et al., Methods Biochem. Anal., 33:337-462 (1988); Anselem, et al., Liposome Technology, CRC Press (1993)). Liposomal formulations can delay clearance and increase cellular uptake (See Reddy, Ann. Pharmacother., 34(7-8):915-923 (2000)). An active agent can also be loaded into a particle prepared from pharmaceutically acceptable ingredients including, but not limited to, soluble, insoluble, permeable, impermeable, biodegradable or gastroretentive polymers or liposomes. Such particles include, but are not limited to, nanoparticles, biodegradable nanoparticles, microparticles, biodegradable microparticles, nanospheres, biodegradable nanospheres, microspheres, biodegradable microspheres, capsules, emulsions, liposomes, micelles and viral vector systems.

The carrier can also be a polymer, e.g., a biodegradable, biocompatible polymer matrix. In one embodiment, the therapeutic peptide can be embedded in the polymer matrix, while maintaining protein integrity. The polymer may be natural, such as polypeptides, proteins or polysaccharides, or synthetic, such as poly α-hydroxy acids. Examples include carriers made of, e.g., collagen, fibronectin, elastin, cellulose acetate, cellulose nitrate, polysaccharide, fibrin, gelatin, and combinations thereof. In one embodiment, the polymer is poly-lactic acid (PLA) or copoly lactic/glycolic acid (PGLA). The polymeric matrices can be prepared and isolated in a variety of forms and sizes, including microspheres and nanospheres. Polymer formulations can lead to prolonged duration of therapeutic effect. (See Reddy, Ann. Pharmacother., 34(7-8):915-923 (2000)). A polymer formulation for human growth hormone (hGH) has been used in clinical trials. (See Kozarich and Rich, Chemical Biology, 2:548-552 (1998)).

Examples of polymer microsphere sustained release formulations are described in PCT publication WO 99/15154 (Tracy, et al.), U.S. Pat. Nos. 5,674,534 and 5,716,644 (both to Zale, et al.), PCT publication WO 96/40073 (Zale, et al.), and PCT publication WO 00/38651 (Shah, et al.). U.S. Pat. Nos. 5,674,534 and 5,716,644 and PCT publication WO 96/40073 describe a polymeric matrix containing particles of erythropoietin that are stabilized against aggregation with a salt.

In some embodiments, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using known techniques. The materials can also be obtained commercially, e.g., from Alza Corporation and Nova Pharmaceuticals, Inc. Liposomal suspensions (including liposomes targeted to specific cells with monoclonal antibodies to cell-specific antigens) can also be used as pharmaceutically acceptable carriers. These can be prepared according to methods known to those skilled in the art, for example, as described in U.S. Pat. No. 4,522,811.

The therapeutic compounds can also be formulated to enhance intracellular delivery. For example, liposomal delivery systems are known in the art, see, e.g., Chonn and Cullis, “Recent Advances in Liposome Drug Delivery Systems,” Current Opinion in Biotechnology 6:698-708 (1995); Weiner, “Liposomes for Protein Delivery: Selecting Manufacture and Development Processes,” Immunomethods, 4(3):201-9 (1994); and Gregoriadis, “Engineering Liposomes for Drug Delivery: Progress and Problems,” Trends Biotechnol., 13(12):527-37 (1995). Mizguchi, et al., Cancer Lett., 100:63-69 (1996), describes the use of fusogenic liposomes to deliver a protein to cells both in vivo and in vitro.

Dosage, toxicity and therapeutic efficacy of any therapeutic agent can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are advantageous. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds may be within a range of circulating concentrations that include the ED50 with little or no toxicity. The dosage may vary within this range depending upon the dosage form employed and the route of administration utilized. For any compound used in the methods, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to determine useful doses in humans accurately. Levels in plasma may be measured, for example, by high performance liquid chromatography.

Typically, an effective amount of the aromatic-cationic peptides, sufficient for achieving a therapeutic or prophylactic effect, ranges from about 0.000001 mg per kilogram body weight per day to about 10,000 mg per kilogram body weight per day. Suitably, the dosage ranges are from about 0.0001 mg per kilogram body weight per day to about 100 mg per kilogram body weight per day. For example dosages can be 1 mg/kg body weight or 10 mg/kg body weight every day, every two days or every three days or within the range of 1-10 mg/kg every week, every two weeks or every three weeks. In one embodiment, a single dosage of peptide ranges from 0.001-10,000 micrograms per kg body weight. In one embodiment, aromatic-cationic peptide concentrations in a carrier range from 0.2 to 2000 micrograms per delivered milliliter. An exemplary treatment regime entails administration once per day or once a week. In therapeutic applications, a relatively high dosage at relatively short intervals is sometimes required until progression of the disease is reduced or terminated, or until the subject shows partial or complete amelioration of symptoms of disease. Thereafter, the patient can be administered a prophylactic regime.

In some embodiments, a therapeutically effective amount of an aromatic-cationic peptide may be defined as a concentration of peptide at the target tissue of 10⁻¹² to 10⁻⁶ molar, e.g., approximately 10⁻⁷ molar. This concentration may be delivered by systemic doses of 0.001 to 100 mg/kg or equivalent dose by body surface area. The schedule of doses would be optimized to maintain the therapeutic concentration at the target tissue, such as by single daily or weekly administration, but also including continuous administration (e.g., parenteral infusion or transdermal application).

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

The mammal treated in accordance with the present methods can be any mammal, including, for example, farm animals, such as sheep, pigs, cows, and horses; pet animals, such as dogs and cats; laboratory animals, such as rats, mice and rabbits. In some embodiments, the mammal is a human.

Combination Therapy with Aromatic-Cationic Peptides

In some embodiments, the aromatic-cationic peptides, such as 2′6′-Dmt-D-Arg-Phe-Lys-NH₂, Phe-D-Arg-Phe-Lys-NH₂, or D-Arg-2′6′-Dmt-Lys-Phe-NH₂, or a pharmaceutically acceptable salt thereof, such as acetate, tartrate, or trifluoroacetate salt, may be combined with one or more additional therapies for the prevention or treatment of PAH. Additional therapeutic agents include, but are not limited to, one or more additional therapeutic agents selected from the group consisting of: endothelin receptor antagonists (ETRAs), guanylate cyclase stimulators, prostacyclin analogues, phosphodiesterase (PDE)-5 inhibitors, dehydroepiandrosterone (DHEA), cyclosporine, tacrolimums, bestatin, imatinib, calcium-channel blockers (CCBs), dichloroacetate (DCA), trimetazidine, ranolazine, 4-phenylbutyrate, tauroursodeoxycholic acid, and salubrinal.

In some embodiments, the endothelin receptor antagonists (ERTAs) are selected from the group consisting of bosentan, ambrisentan, macitentan, and sitaxsentan. In some embodiments, the prostacyclin analogues are selected from the group consisting of epoprostenal, treprostinil, and iloprost. In some embodiments, the soluble guanylate cyclase stimulators are selected from the group consisting of riociguat and cinaciguat. In some embodiments, the phosphodiesterase (PDE)-5 inhibitors are selected from the group consisting of sildenafil, tadalafil, and vardenafil. In some embodiments, the calcium-channel blockers (CCBs) are selected from the group consisting of nifedipine, diltiazem, and amlodipine.

In one embodiment, an additional therapeutic agent is administered to a subject in combination with an aromatic cationic peptide, such that a synergistic therapeutic effect is produced. For example, administration of the peptide with one or more additional therapeutic agents for the prevention or treatment of PAH will have greater than additive effects in the prevention or treatment of the disease. Therefore, lower doses of one or more of any individual therapeutic agent may be used in treating or preventing PAH, resulting in increased therapeutic efficacy and decreased side-effects. In some embodiments, the aromatic-cationic peptide is administered in combination with one or more additional therapeutic agents selected from the group consisting of endothelin receptor antagonists (ETRAs), guanylate cyclase stimulators, prostacyclin analogues, phosphodiesterase (PDE)-5 inhibitors, dehydroepiandrosterone (DHEA), cyclosporine, tacrolimums, bestatin, imatinib, calcium-channel blockers (CCBs), dichloroacetate (DCA), trimetazidine, ranolazine, 4-phenylbutyrate, tauroursodeoxycholic acid, and salubrinal, such that a synergistic effect in the prevention or treatment of PAH results.

In any case, the multiple therapeutic agents may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may vary from more than zero weeks to less than four weeks. In addition, the combination methods, compositions and formulations are not to be limited to the use of only two agents.

### EXAMPLES

The present technology is further illustrated by the following examples, which should not be construed as limiting in any way. For each of the examples below, any aromatic-cationic peptide described herein could be used. By way of example, but not by limitation, the aromatic-cationic peptide used in the example below could be 2′6′-Dmt-D-Arg-Phe-Lys-NH₂, Phe-D-Arg-Phe-Lys-NH₂, or D-Arg-2′6′-Dmt-Lys-Phe-NH₂ or any one or more of the peptides shown in Tables A, B, C, D, and/or E.

### Example 1

### Use of Aromatic-Cationic Peptides in the Treatment of Pulmonary Arterial Hypertension (PAH) in a Mouse Model

This Example demonstrates the use of aromatic-cationic peptides, such as 2′6′-Dmt-D-Arg-Phe-Lys-NH₂, Phe-D-Arg-Phe-Lys-NH₂, or D-Arg-2′6′-Dmt-Lys-Phe-NH₂, or a pharmaceutically acceptable salt thereof, such as acetate, tartrate, or trifluoroacetate salt, in the treatment of pulmonary arterial hypertension (PAH) in a mouse model. For this example, L1cre(+);Bmpr2^{f/f} mice, as described by Hong, et al. (Circulation 118:722-730 (2008)), will be used. These mice, which are characterized by a genetic ablation of Bmpr2 in pulmonary endothelial cells, exhibit elevated right ventricular systolic pressure, right ventricular hypertrophy, and histopathological features reminiscent of human PAH lungs.

### Methods

Three-month-old male L1cre(+);Bmpr2^{f/f} mice are randomly assigned to either a sham (untreated) group or aromatic-cationic peptide-treated group (L1 cre(+);Bmpr2^{f/f}-peptide). Mice in the L1cre(+);Bmpr2^{f/f}-peptide group are given a daily intraperitoneal injection of peptide (1-16 mg/kg), whereas the sham group will receive a daily intraperitoneal injection of vehicle. Treatments will continue until the day of sacrifice.

Hemodynamic analysis. Systemic blood pressure is recorded noninvasively using the tail-cuff method. A pneumatic pulse sensor is placed on the tail distal to an occlusion cuff controlled by a Programmed Electro-Sphygmomanometer (PE-300, Narco Bio-Systems, TX), which is connected to the Powerlab system (ADinstrument, CO). To evaluate pulmonary artery pressure, right ventricular systolic pressure (RVSP) is measured by right heart catheterization (RHC) through the right jugular vein. Briefly, each mouse is anesthetized by ketamine (100 mg/kg) and xylazine (15 mg/kg) and placed with supine position. A 1-2 cm incision is made to expose the right jugular vein. A Mikro-Tip pressure transducer (SPR-835, Millar Instrument, TX) is inserted into right external jugular vein and advanced into the right ventricle. All electrical outputs from the tail cuff, the pulse sensor, and transducer are recorded and analyzed by Powerlab 8/30 data acquisition system and associated Chart software (ADinstrument, CO).

Pulmonary vessel morphometry. After hemodynamic analysis, mice are euthanized, and organs including the heart and lungs are isolated for further analyses. Using syringe-generated flow, the pulmonary circulation is perfused with PBS containing heparin (3 units/mL). Outflow tract and atria are removed prior to measuring the weights of the right ventricle and left ventricle plus septum. The left lung is inflated with PBS for 20 minutes followed by formalin at constant inflation pressure of 23 cmH₂O and embedded in paraffin. Each lung sample is transversely sectioned in 5 μm thickness and subjected to hematoxylin and eosin (H&E) staining and immunostaining with antibody against smooth muscle α-actin (αSMA, clone: 1A4; Sigma-Aldrich, 1:800). In each section, αSMA-positive vessels are categorized by their locations, such as vessels at the level of terminal bronchioli, respiratory bronchioli, alveolar ducts, or alveolar sac. To assess muscularization of pulmonary vessels, peripheral blood vessels ranging from 30-70 μm in diameter are counted in at least four fields at 20× magnification with a Zeiss Axioplan-2 optical microscope. The counted vessels are categorized as fully muscularized (75-100% of medial layer covered by anti-αSMA staining), partially muscularized (1-74% of medial layer is covered by anti-αSMA staining), or nonmuscularized vessels at the level of alveolar ducts. The percentage of pulmonary vessels in each category is calculated by dividing the number of vessels in the category by the total number of counted vessels in the same field. To calculate percentage of wall thickness (WT), circular and fully muscularized vessels ranging from 30-70 μm in diameter are selected. WT¹ (the thickness between the outer boundary and the inner boundary of αSMA positive medial layer) is measured at one point of the vessel wall and WT² at the point which was diametrically opposite, guided by Openlab 5.03 Beta software (Improvision, Inc., Lexington, Mass.). External diameter (ED) is also measured at the same vessel. The percentage medial wall thickness for these vessels is calculated as (WT¹+WT²)×100/ED.

### Results

It is predicted that the untreated L1cre(+);Bmpr2^{f/f} mice will exhibit significantly increased right ventricular systolic pressure values and right ventricular hypertrophy as measured by the ratio of RV to left ventricle plus septum, as compared with age-matched wild-type mice. It is also predicted that immunostaining of lung sections obtained from untreated L1cre(+);Bmpr2^{f/f} mice with anti-a-smooth muscle actin (aSMA) antibodies will reveal an increased number of and increased wall thickness of αSMA-positive small arteries as compared with age-matched wild-type mice. It is also anticipated that untreated L1cre(+);Bmpr2^{f/f} mice will exhibit vascular lesions characterized by a thickening of αSMA-positive cell layers in pulmonary arteries with some of the arteries appearing occluded, resembling the concentric vascular lesion in human PAH lung samples.

Treatment with aromatic-cationic peptides, such as 2′6′-Dmt-D-Arg-Phe-Lys-NH₂, Phe-D-Arg-Phe-Lys-NH₂, or D-Arg-2′6′-Dmt-Lys-Phe-NH₂, or pharmaceutically acceptable salts thereof, such as acetate, tartrate, or trifluoroacetate salt, is anticipated to significantly reduce right ventricular systolic pressures and right ventricular hypertrophy in L1cre(+);Bmpr2^{f/f}-peptide mice relative to the untreated L1cre(+);Bmpr2^{f/f} mice. It is also anticipated that L1cre(+);Bmpr2^{f/f}-peptide mice will exhibit a decreased number of and decreased wall thickness of αSMA-positive small arteries as compared to untreated L1cre(+);Bmpr2^{f/f} mice. It is further expected that L1cre(+);Bmpr2^{f/f}-peptide mice will exhibit decreased vascular lesions relative to untreated L1cre(+);Bmpr2^{f/f} mice.

These results will show that aromatic-cationic peptides, such as 2′6′-Dmt-D-Arg-Phe-Lys-NH₂, Phe-D-Arg-Phe-Lys-NH₂, or D-Arg-2′6′-Dmt-Lys-Phe-NH₂, or a pharmaceutically acceptable salt thereof, such as acetate, tartrate, or trifluoroacetate salt are useful in the treatment of PAH in a mouse model. Accordingly, the peptides are useful in methods comprising administering aromatic-cationic peptides to a subject in need thereof for the treatment of PAH.

### Example 2

### Use of Aromatic-Cationic Peptides in the Treatment of Pulmonary Arterial Hypertension (PAH) in a Rat Monocrotaline Model

This Example demonstrates the use of aromatic-cationic peptides, such as 2′6′-Dmt-D-Arg-Phe-Lys-NH₂, Phe-D-Arg-Phe-Lys-NH₂, or D-Arg-2′6′-Dmt-Lys-Phe-NH₂, or a pharmaceutically acceptable salt thereof, such as acetate, tartrate, or trifluoroacetate salt, in the treatment of pulmonary arterial hypertension (PAH) in a monocrotaline-treated rat model. The monocrotaline model (Werchan, et al, Am. J. Physiol. 256:H1328-1336 (1989)) is considered as a standard model for idiopathic PAH (IPAH).

### Methods

MCT Treatment. Monocrotaline (MCT) is dissolved in 0.5 N of HCl, and the pH is adjusted to 7.4 with 0.5 N of NaOH. The solution is administered as a single subcutaneous injection (60 mg/kg) to eight-week-old male Sprague Dawley rats. Control rats received an equal volume (3mL/kg) of isotonic saline. The MCT-treated rats are randomly assigned to either a sham (untreated) group or aromatic-cationic peptide-treated group (MCT-peptide). Rats in the MCT-peptide group are given a daily intraperitoneal injection of peptide (1-16 mg/kg), whereas the sham group will receive a daily intraperitoneal injection of vehicle. Treatments will continue until the day of sacrifice.

The planned observation time is 42 days. MCT-injected rats are to be killed if they develop clinical signs of right ventricular (RV) failure and/or appear clinically to be severely stressed. Such a condition is defined by the occurrence of a body weight (BW) loss of more than 30 g in the preceding week or more than 15 g in the preceding 4 days, in combination with at least one of the following criteria: (1) dyspnea, defined as visibly increased respiratory efforts and chest-opposite-to-belly breath movement; (2) cold lower body, tail, and limb temperature assessed subjectively by physical examination; (3) cyanotic ears; and (4) markedly decreased activity level (lethargy).

Surgical Preparation and Tissue Preparation. The animals are initially anesthetized with intraperitoneal pentobarbital and ventilated with 10 mL/kg body weight and a frequency of 60 s⁻¹ (SAR⁸³⁰A/P; IITC, Woodland Hills, Calif.) after tracheostomy. Anesthesia is maintained by inhalation of isoflurane.

Hemodynamic Measurements. A right heart catheter (PE 50 tubing) is inserted through the right jugular vein for measurement of right ventricular pressure, and the left carotid artery is cannulated for arterial pressure monitoring. Cardiac output is measured by thermodilution technique (Cardiotherm 500-X; Hugo-Sachs Electronic-Harvard Apparatus GmbH, March-Hugstetten, Germany). Briefly, a thermistor catheter is forwarded into the ascending thoracic aorta via the right carotid artery for the measurement of transpulmonary thermodilution cardiac output. A 0.15-mL bolus of room-temperature saline is injected into the right ventricle as an indicator, and cardiac output is averaged from three consecutive determinations and indexed to the weight of the animal to obtain the cardiac index. After exsanguinations, the left lung is fixed for histology in 10% neutral-buffered formalin, and the right lung is frozen in nitrogen.

Measurement of Organ Weight. The heart is dissected, and the ratio of the right ventricle to left ventricle plus septum weight (RV/LV+S) is calculated as an index of right ventricular hypertrophy.

Paraffin Embedding and Microscopy. Fixation is performed by immersion of the lungs in a 3% paraformaldehyde solution. For paraffin embedding, whole lung is dissected in tissue blocks from all lobes. Sectioning at 10 μm is performed from all paraffin-embedded blocks. Hematoxylin and eosin elastica staining is performed according to common histopathologic procedures. Light microscopic slides are analyzed in a blind fashion without the knowledge of treatment groups. In each rat, 40 to 50 intraacinar arteries are categorized as muscular (i.e., with a complete medial coat of muscle), partially muscular (i.e., with only a crescent of muscle), or nonmuscular (i.e., no apparent muscle). Microscopy and photography are performed with a Nikon UFX-II microscope with a Nikon D1 attached to the phototube at a magnification of ×100-×400.

### Results

It is predicted that the untreated MCT-rats will exhibit significantly increased right ventricular systolic pressure values, right ventricular hypertrophy, and pulmonary vascular resistance as compared with age-matched saline-injected control animals. It is also predicted that cardiac output will be reduced in untreated MCT-rats as compared with age-matched saline-injected control animals. It is also anticipated that untreated MCT-rats will exhibit increased medial hypertrophy of the pulmonary arteries and distal pulmonary artery muscularization compared with age-matched saline-injected control animals.

Treatment with aromatic-cationic peptides, such as 2′6′-Dmt-D-Arg-Phe-Lys-NH₂, Phe-D-Arg-Phe-Lys-NH₂, or D-Arg-2′6′-Dmt-Lys-Phe-NH₂, or pharmaceutically acceptable salts thereof, such as acetate, tartrate, or trifluoroacetate salt, is anticipated to significantly reduce right ventricular systolic pressures, right ventricular hypertrophy, and pulmonary vascular resistance in MCT-peptide rats relative to the untreated MCT-rats. It is also anticipated that cardiac output will be improved in MCT-peptide rats relative to untreated MCT-rats. In is further expected that MCT-rats will exhibit decreased medial hypertrophy of the pulmonary arteries and reduced distal pulmonary artery muscularization relative to untreated MCT-rats.

These results will show that aromatic-cationic peptides, such as 2′6′-Dmt-D-Arg-Phe-Lys-NH₂, Phe-D-Arg-Phe-Lys-NH₂, or D-Arg-2′6′-Dmt-Lys-Phe-NH₂, or a pharmaceutically acceptable salt thereof, such as acetate, tartrate, or trifluoroacetate salt are useful in the treatment of PAH in a rat model. Accordingly, the peptides are useful in methods comprising administering aromatic-cationic peptides to a subject in need thereof for the treatment of PAH.

### Example 3

### Use of Aromatic-Cationic Peptides in the Treatment of Pulmonary Arterial Hypertension (PAH) in Humans

This Example demonstrates the use of aromatic-cationic peptides, such as 2′6′-Dmt-D-Arg-Phe-Lys-NH₂, Phe-D-Arg-Phe-Lys-NH₂, or D-Arg-2′6′-Dmt-Lys-Phe-NH₂, or a pharmaceutically acceptable salt thereof, such as acetate, tartrate, or trifluoroacetate salt, in the treatment of PAH.

### Methods

Subjects suspected of having or diagnosed as having PAH receive daily administrations of 1%, 5%, or 10% solution of aromatic-cationic peptide, such as 2′6′-Dmt-D-Arg-Phe-Lys-NH₂, Phe-D-Arg-Phe-Lys-NH₂, or D-Arg-2′6′-Dmt-Lys-Phe-NH₂, or a pharmaceutically acceptable salt thereof, such as acetate, tartrate, or trifluoroacetate salt, alone or in combination with one or more additional therapeutic agents for the treatment or prevention of PAH. Additional therapeutic agents selected from the group consisting of: endothelin receptor antagonists (ETRAs), guanylate cyclase stimulators, prostacyclin analogues, phosphodiesterase (PDE)-5 inhibitors, dehydroepiandrosterone (DHEA), cyclosporine, tacrolimums, bestatin, imatinib, calcium-channel blockers (CCBs), dichloroacetate (DCA), trimetazidine, ranolazine, 4-phenylbutyrate, tauroursodeoxycholic acid, and salubrinal are administered orally, topically, systemically, intravenously, subcutaneously, intraperitoneally, intramuscularly, or by inhalation according to methods known in the art. Dosages of the one or more additional therapeutic agents will range between 0.1 mg/kg to 50 mg/kg. Subjects will be evaluated weekly for the presence and/or severity of signs and symptoms associated with PAH, including, but not limited to, e.g., persistent dyspnea on exertion, chest pain, light-headedness, exertional presyncope/syncope, palpitations, fatigue, weakness, hoarseness in the voice due to compression of the left laryngeal nerve by the dilated pulmonary artery, venous jugular distension, hepato jugular reflux, hepatomegaly, hepatalgia, lower limb edema, ascites, generalized edema, intimal fibrosis of pulmonary arteries, increased medial thickness of pulmonary arteries, intimal hyperplasia of muscular pulmonary arteries, pulmonary artery thrombotic lesions, pulmonary arteriolar occlusion, pulmonary vascular pruning, plexiform lesions in pulmonary arteries, elevated serum or plasma brain natriuretic peptide (BNP) (>180 pg/mL), and elevated serum or plasma N-terminal fragment of proBNP (NT-proBNP) (≥1400 pg/mL). Treatments are maintained until such a time as one or more signs or symptoms of PAH are ameliorated or eliminated.

Methods for assessing the signs, symptoms, or complications of PAH are known in the art. Exemplary methods for assaying the signs, symptoms, or complications of PAH include, but are not limited to, e.g., the 6-minute walk test (6MWT), right heart catheterization (RHC) to measure mean pulmonary arterial pressure (mPAP) and pulmonary wedge pressure, serum or plasma brain natriuretic peptide (BNP) and/or N-terminal fragment of proBNP (NT-proBNP) measurements performed using standard laboratory techniques, Doppler echocardiography, cardiac magnetic resonance imaging (MRI), and angiography.

### Results

It is predicted that subjects suspected of having or diagnosed as having PAH and receiving therapeutically effective amounts of aromatic-cationic peptide, such as 2′6′-Dmt-D-Arg-Phe-Lys-NH₂, Phe-D-Arg-Phe-Lys-NH₂, or D-Arg-2′6′-Dmt-Lys-Phe-NH₂, or a pharmaceutically acceptable salt thereof, such as acetate, tartrate, or trifluoroacetate salt will display reduced severity or elimination of symptoms associated with PAH. It is also expected that PAH subjects treated with the aromatic-cationic peptide will show normalization of BNP and/or NT-proBNP blood levels by at least 5% compared to the untreated PAH controls. It is further expected that administration of 2′6′-Dmt-D-Arg-Phe-Lys-NH₂, Phe-D-Arg-Phe-Lys-NH₂, or D-Arg-2′6′-Dmt-Lys-Phe-NH₂ in combination with one or more additional therapeutic agents will have synergistic effects in this regard compared to that observed in subjects treated with the aromatic-cationic peptides or the additional therapeutic agents alone.

These results will show that aromatic-cationic peptides, such as 2′6′-Dmt-D-Arg-Phe-Lys-NH₂, Phe-D-Arg-Phe-Lys-NH₂, or D-Arg-2′6′-Dmt-Lys-Phe-NH₂, or a pharmaceutically acceptable salt thereof, such as acetate, tartrate, or trifluoroacetate salt are useful in the treatment of PAH. These results will show that aromatic-cationic peptides, such as 2′6′-Dmt-D-Arg-Phe-Lys-NH₂, Phe-D-Arg-Phe-Lys-NH₂, or D-Arg-2′6′-Dmt-Lys-Phe-NH₂, or a pharmaceutically acceptable salt thereof, such as acetate, tartrate, or trifluoroacetate salt are useful in ameliorating one or more of the following symptoms: persistent dyspnea on exertion, chest pain, light-headedness, exertional presyncope/syncope, palpitations, fatigue, weakness, hoarseness in the voice due to compression of the left laryngeal nerve by the dilated pulmonary artery, venous jugular distension, hepato jugular reflux, hepatomegaly, hepatalgia, lower limb edema, ascites, generalized edema, intimal fibrosis of pulmonary arteries, increased medial thickness of pulmonary arteries, intimal hyperplasia of muscular pulmonary arteries, pulmonary artery thrombotic lesions, pulmonary arteriolar occlusion, pulmonary vascular pruning, plexiform lesions in pulmonary arteries, elevated serum or plasma brain natriuretic peptide (BNP) (>180 pg/mL), and elevated serum or plasma N-terminal fragment of proBNP (NT-proBNP) (≥1400 pg/mL). Accordingly, the peptides are useful in methods comprising administering aromatic-cationic peptides to a subject in need thereof for the treatment of PAH.

### Example 4

### Use of Aromatic-Cationic Peptides in the Treatment of Pulmonary Arterial Hypertension (PAH) in Humans

This Example demonstrates the use of D-Arg-2′6′-Dmt-Lys-Phe-NH₂ or a pharmaceutically acceptable salt thereof, such as acetate, tartrate, or trifluoroacetate salt, in the treatment of PAH.

### Methods

A single-center, randomized, double-blind, placebo-controlled, single ascending dose trial of D-Arg-2′6′-Dmt-Lys-Phe-NH₂ was performed in heart failure subjects aged 45-80 years (n=36) with reduced ejection fraction (HFrEF≤35%). Three sequential dose-escalation cohorts were enrolled and administered a single intravenous (IV) dose of D-Arg-2′6′-Dmt-Lys-Phe-NH₂ by a 4-hour infusion. In each cohort, 8 subjects were randomly assigned to receive D-Arg-2′6′-Dmt-Lys-Phe-NH₂ (active group) and 4 to receive placebo (placebo group infused with 0.9% saline solution in the same manner as the D-Arg-2′6′-Dmt-Lys-Phe-NH₂ active group). Eligible subjects were admitted to the research unit on Day 1, were randomized and received the study drug infusion on Day 2 and were discharged on Day 3. Subjects returned to the research unit on Day 7 (+3) for follow-up. The dose levels of D-Arg-2′6′-Dmt-Lys-Phe-NH₂ in Cohorts 1, 2, and 3 were 0.005, 0.05, and 0.25 mg/kg/hour, respectively.

Safety assessments, including 12-lead electrocardiograms (ECGs), clinical laboratory, vital signs, full and symptom-directed physical examinations, and recording of adverse events, were performed daily during the admission (Days 1-3) and at the End-Of-Study Visit on Day 7. Blood samples for pharmacokinetic determinations of D-Arg-2′6′-Dmt-Lys-Phe-NH₂ and its metabolites were collected during Day 2 and Day 3.

A single dose of the aromatic-cationic peptide, D-Arg-2′6′-Dmt-Lys-Phe-NH₂, was administered intravenously (IV) at 0.005 mg/kg/hour (Cohort 1; low dose), 0.05 mg/kg/hour (Cohort 2; intermediate dose), and 0.25 mg/kg/hour (Cohort 3; high dose) over 4 hours at 60 mL/hour.

Echocardiograms (2-D) were performed on Day 2 at pre-dose (0-hr), mid-dose (2-hr), end-dose (4-hr), and 6 hours, 8 hours, 12 hours after infusion initiation, and at Day 3 at 24 hours after infusion initiation in every subject. Primary echo endpoints included end-diastolic volume (EDV) and EDV index, and end-systolic volume (ESV) and ESV index. Secondary echo endpoints included left atrial (LA) volume, right ventricular fractional area change (RV FAC), and right ventricular systolic pressure (RVSP). Exploratory echo endpoints included global longitudinal strain.

Safety measures included the assessments and characterization of the type, incidence, severity, seriousness, and relationship to treatment of adverse events (AEs); effects on vital signs and laboratory parameters; and changes in ECGs and physical examinations from baseline.

All statistical analyses, summary tables and data listings were prepared using SAS software Version 9.2 or higher. Overall differences in pharmacokinetic parameters of systemic exposure (e.g., Cmax and AUC) across the 3 different dosing regimens were evaluated by ANOVA. In general, significance was determined at the p<0.05 level. For continuous variables, descriptive statistics (sample size, mean, standard deviation, median, minimum, and maximum values) were determined. For discrete variables, frequency distributions were generated.

### Results

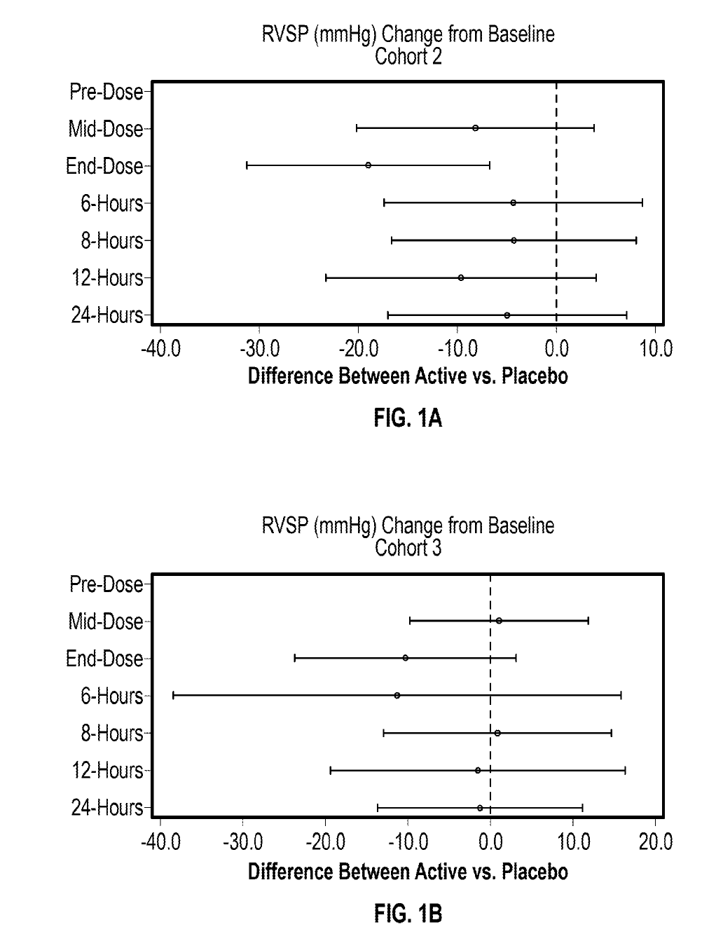

As shown in Table 5 and FIG. 1A, Cohort 2 subjects treated with 0.05 mg/kg/hour D-Arg-2′6′-Dmt-Lys-Phe-NH₂ exhibit a significant decrease in pulmonary pressures (RVSP) at end dose relative to baseline measurements. A similar trend is shown in Cohort 3 subjects treated with 0.25 mg/kg/hour D-Arg-2′6′-Dmt-Lys-Phe-NH₂ (Table 6 and FIG. 1B).

TABLE 5
Cohort 2 RVSP (mmHg): Δ from baseline

| | | Mean Active | | Mean Pooled | | Mean | | |
|---|---|---|---|---|---|---|---|---|
| | | Group with | | Placebo with | | Difference with | | |
| Time Point | | 95% CI | | 95% CI | | 95% CI | | P-value |
| Mid-Dose | | −8.5 | (−17.9, 1.0) | −0.2 | (−7.6, 7.1) | −8.2 | (−20.2, 3.8) | 0.1441 |
| End-Dose | | −9.9 | (−18.6, −1.3) | 9.1 | (0.4, 17.8) | −19.1 | (−31.3, −6.8) | 0.0090 |
| 6 | Hours | −4.8 | (−14.5, 5.0) | −0.4 | (−9.1, 8.4) | −4.4 | (−17.5, 8.7) | 0.4516 |
| 8 | Hours | −6.5 | (−15.2, 2.2) | −2.1 | (−10.8, 6.6) | −4.4 | (−16.7, 8.0) | 0.4188 |
| 12 | Hours | −5.6 | (−15.3, 4.0) | 4.1 | (−5.6, 13.7) | −9.7 | (−23.4, 3.9) | 0.1320 |
| 24 | Hours | −5.3 | (−13.9, 3.2) | −0.3 | (−8.8, 8.2) | −5.0 | (−17.1, 7.1) | 0.3479 |

TABLE 6
Cohort 3 RVSP (mmHg): Δ from baseline

| | | Mean Active | | Mean Pooled | | Mean | | |
|---|---|---|---|---|---|---|---|---|
| | | Group with | | Placebo with | | Difference with | | |
| Time Point | | 95% CI | | 95% CI | | 95% CI | | P-value |
| Mid-Dose | | 0.9 | (−6.4, 8.1) | −0.2 | (−8.2, 7.7) | 1.1 | (−9.7, 11.9) | 0.8245 |
| End-Dose | | −1.2 | (−10.1, 7.8) | 9.1 | (−0.9, 19.1) | −10.3 | (−23.7, 3.1) | 0.1121 |
| 6 | Hours | −11.7 | (−36.4, 13.1) | −0.4 | (−11.4, 10.7) | −11.3 | (−38.5, 15.8) | 0.3102 |
| 8 | Hours | −1.3 | (−11.7, 9.2) | −2.1 | (−11.2, 6.9) | 0.9 | (−13.0, 14.7) | 0.8789 |
| 12 | Hours | 2.6 | (−11.9, 17.1) | 4.1 | (−6.2, 14.4) | −1.5 | (−19.3, 16.3) | 0.8280 |
| 24 | Hours | −1.6 | (−10.4, 7.2) | −0.3 | (−9.1, 8.5) | −1.3 | (−13.7, 11.2) | 0.8085 |

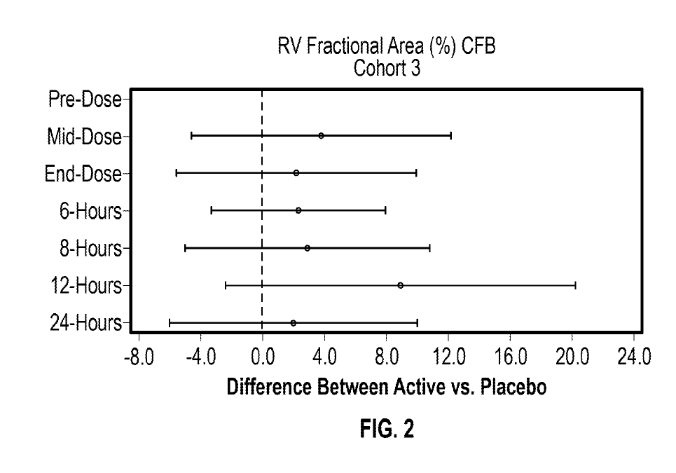

As shown in Table 7 and FIG. 2, Cohort 3 subjects treated with 0.25 mg/kg/hour D-Arg-2′6′-Dmt-Lys-Phe-NH₂ exhibit an overall improvement in right ventricular fractional area change (RV FAC).

TABLE 7
Cohort 3 RV FAC (%): Δ from baseline

| | | Mean Active | Mean Pooled | | Mean | | |
|---|---|---|---|---|---|---|---|
| | | Group with | Placebo with | | Difference with | | |
| Time Point | | 95% CI | 95% CI | | 95% CI | | P-value |
| Mid-Dose | | 1.6 (−2.6, 5.8) | −2.2 | (−9.4, 5.1) | 3.8 | (−4.6, 12.2) | 0.3094 |
| End-Dose | | 0.3 (−4.8, 5.5) | 0.0 | (−6.3, 6.3) | 0.3 | (−7.8, 8.4) | 0.9341 |
| 6 | Hours | 2.6 (−0.7, 5.9) | −1.2 | (−5.3, 2.8) | 3.8 | (−1.4, 9.0) | 0.1299 |
| 8 | Hours | 3.4 (−0.7, 7.5) | −1.2 | (−8.3, 5.9) | 4.6 | (−3.6, 12.8) | 0.2191 |
| 12 | Hours | 1.7 (−4.0, 7.4) | −7.2 | (−17.0, 2.7) | 8.9 | (−2.5, 20.2) | 0.1040 |
| 24 | Hours | 2.5 (−2.0, 7.0) | −1.3 | (−8.2, 5.5) | 3.8 | (−4.3, 12.0) | 0.3106 |

These results show that compositions comprising aromatic-cationic peptides, such as D-Arg-2′6′-Dmt-Lys-Phe-NH₂, are useful in decreasing RVSP and improving RV FAC in human subjects. Accordingly, the peptides are useful in methods comprising administering aromatic-cationic peptides to a subject in need thereof for the treatment of PAH.

### Equivalents

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the appended claims. The present technology is to be limited only by the terms of the appended claims, along with the full scope of equivalents to which such claims are entitled. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

Other embodiments are set forth within the following claims.

What is claimed is:

1. A method for treating or preventing pulmonary arterial hypertension (PAH) in a subject in need thereof, comprising administering to the subject a therapeutically effective amount of the peptide D-Arg-2′,6′-Dmt-Lys-Phe-NH₂ or a pharmaceutically acceptable salt thereof, thereby resulting in the treatment or prevention of one or more signs or symptoms of PAH.

2. The method of claim 1, wherein the subject displays elevated blood levels of brain natriuretic peptide (BNP) and/or N-terminal fragment of proBNP (NT-proBNP) compared to a normal control subject, and wherein peptide administration normalizes blood levels of BNP and/or NT-proBNP.

3. The method of any one of claim 1 or 2, wherein the subject has been diagnosed as having PAH.

4. The method of claim 3, wherein the signs or symptoms of PAH comprise one or more of persistent dyspnea on exertion, chest pain, light-headedness, exertional presyncope/syncope, palpitations, fatigue, weakness, hoarseness in the voice due to compression of the left laryngeal nerve by the dilated pulmonary artery, venous jugular distension, hepato jugular reflux, hepatomegaly, hepatalgia, lower limb edema, ascites, generalized edema, intimal fibrosis of pulmonary arteries, increased medial thickness of pulmonary arteries, intimal hyperplasia of muscular pulmonary arteries, pulmonary artery thrombotic lesions, pulmonary arteriolar occlusion, pulmonary vascular pruning, plexiform lesions in pulmonary arteries, elevated serum or plasma brain natriuretic peptide (BNP) (>180 pg/mL), and elevated serum or plasma N-terminal fragment of proBNP (NT-proBNP) (≥1400 pg/mL).

5. The method of any one of claims 1-4, wherein the subject is human.

6. The method of any one of claims 1-5, wherein the peptide is administered orally, topically, systemically, intravenously, subcutaneously, transdermally, iontophoretically, intranasally, intraperitoneally, intramuscularly, or by pulmonary inhalation.

7. The method of any one of claims 1-6, further comprising separately, sequentially or simultaneously administering an additional therapeutic agent to the subject.

8. The method of claim 7, wherein the additional therapeutic agent is selected from the group consisting of: endothelin receptor antagonists (ETRAs), guanylate cyclase stimulators, prostacyclin analogues, phosphodiesterase (PDE)-5 inhibitors, dehydroepiandrosterone (DHEA), cyclosporine, tacrolimums, bestatin, imatinib, calcium-channel blockers (CCBs), dichloroacetate (DCA), trimetazidine, ranolazine, 4-phenylbutyrate, tauroursodeoxycholic acid, and salubrinal.

9. The method of claim 8, wherein the combination of the peptide and the additional therapeutic agent has a synergistic effect in the prevention or treatment of PAH.

10. A method for reducing the risk of PAH in a subject in need thereof, the method comprising: administering to the subject a therapeutically effective amount of the peptide D-Arg-2′,6′-Dmt-Lys-Phe-NH₂ or a pharmaceutically acceptable salt thereof, thereby resulting in the prevention or delay of onset of one or more signs or symptoms of PAH.

11. The method of claim 10, wherein the subject harbors a mutation in the bone morphogenetic protein receptor 2 (BMPR²) gene.

12. The method of any one of claims 10-11, wherein the subject is human.

13. The method of any one of claims 10-12, wherein the peptide is administered orally, topically, systemically, intravenously, subcutaneously, transdermally, iontophoretically, intranasally, intraperitoneally, intramuscularly, or by pulmonary inhalation.

14. The method of any one of claims 10-13, further comprising separately, sequentially, or simultaneously administering the additional therapeutic agent to the subject.

15. The method of claim 14, wherein the additional therapeutic agent is selected from the group consisting of: endothelin receptor antagonists (ETRAs), guanylate cyclase stimulators, prostacyclin analogues, phosphodiesterase (PDE)-5 inhibitors, dehydroepiandrosterone (DHEA), cyclosporine, tacrolimums, bestatin, imatinib, calcium-channel blockers (CCBs), dichloroacetate (DCA), trimetazidine, ranolazine, 4-phenylbutyrate, tauroursodeoxycholic acid, and salubrinal.

16. The method of claim 15, wherein the combination of the peptide and the additional therapeutic agent has a synergistic effect in reducing the risk of PAH.
